(19) United States
(12) Patent Application Publication
(10) Pub. No.: US 2011/0142757 A1
(43) Pub. Date: Jun. 16, 2011

(54) NOVEL COMPOUNDS AND THEIR USES IN DIAGNOSIS

(75) Inventors: Michael Kassiou, Bexley (AU); Aaron Reynolds, Glenorie (AU)

(73) Assignee: the University of Sydney

(21) Appl. No.: 13/059,324

(22) PCT Filed: Aug. 19, 2009
(86) PCT No.: PCT/AU09/01063

§ 371 Date: Feb. 16, 2011

(30) Foreign Application Priority Data
Aug. 19, 2008 (AU) 2008904249

(51) Int. Cl. A61K 51/04; C07D 487/04; A61K 31/519; A61K 49/00; A61K 49/04; A61P 35/00; A61P 25/00; A61P 25/28; A61P 25/08; A61P 25/16; A61P 25/22; A61P 9/00; A61P 25/24; A61P 31/00; A61P 31/18; A61P 31/22; A61P 9/10; A61P 29/00

(52) U.S. Cl. 424/1.85; 544/281; 514/259.3; 424/9.1; 424/1.89; 424/9.44

(57) ABSTRACT

A compound of formula (I) wherein, X and Y independently bind TSPO, wherein X and Y are the same or different; and L is a linker that links X to Y; or a salt or solvate thereof. For preference, X and Y may be (II) or (III). The compounds may be radiolabeled with a radioisotope. Also methods for diagnosing or treating TSPO related disorders such as neurodegenerative disorder, inflammation or anxiety, eg. Alzheimer's disease, Parkinson's disease, Huntington's disease, multiple sclerosis, multiple system atrophy, epilepsy, encephalopathy, stroke, brain tumour, anxiety, stress, emotional disturbances or cognitive impairment, glioblastoma, ischemic stroke, herpes encephalitis, HIV, amyotrophic lateral sclerosis, corticobasal degeneration, cancer, depression, an auto-immune disease and an infectious disease.

### FIELD OF THE INVENTION

The present invention relates to novel compounds, processes for their preparation and uses thereof. More specifically, the present invention relates to compounds that bind translocator protein (18 kDa) (TSPO) and methods for imaging TSPO expression in a subject. This invention also relates to methods for the treatment of disorders such as, for example, neurodegenerative disorders, inflammation or anxiety.

### BACKGROUND OF THE INVENTION

Any discussion of the prior art throughout the specification should in no way be considered as an admission that such prior art is widely known or forms part of the common general knowledge in the field.

TSPO, formerly known as the peripheral benzodiazepine receptor (PBR), can form a trimeric complex with the adenine nucleotide carrier (ANC) (30 kDa) and the voltage-dependent anion channel (VDAC) (32 kDa) to constitute the mitochondrial permeability transition pore (MPTP). The TSPO is distinguished from the central benzodiazepine receptor (CBR) by its distinct structure, physiological functions and subcellular location on the outer membrane of the mitochondria. Although the TSPO has been implicated in numerous biological processes, some aspects of its physiological role remain unclear. Studies implicate the TSPO in the rate limiting step of steroid biosynthesis, immunomodulation, porphyrin transport, calcium homeostasis, and programmed cell death.

The TSPO has been implicated in a variety of diseases, including: glioblastoma (Pappata et al., 1991 J Nucl Med 32:1608-10; Veenman et al., 2004 Biochem Pharmacol. 68(4):689-98; Levin, 2005 Biochemistry 44(29):9924-35), multiple sclerosis (Vowinckel et al., 1997 J Neurosci Res 50:345-53; Banati et al., 2000 Brain 123 (Pt 11): 2321-37; Debruyne et al., 2003 Eur J Neurol 10: 257-64; Versijpt et al., 2005 Mult Scler 11:127-34; Chen and Guilarte, 2006 Toxicol Sci. 91(2):532-9), ischemic stroke (Gerhard et al., 2000 Neuroreport; 11:2957-60; Gerhard et al., 2005 Neuroimage 24:591-5; Price et al., 2006 Stroke 37:1749-53), herpes encephalitis (Cagnin et al., 2001 Brain; 124:2014-27), Parkinson's disease (Cumming et al., 2001. Acta Neurol Scand 103:309-15; Cicchetti et al., 2002 Eur J Neurosci 15:991-8; Ouchi et al., 2005 57:168-75; Gerhard et al., 2006 Neurobiol Dis 21:404-12; Cumming et al., 2006 Synapse 59:418-26), HIV (Venneti et al., 2004 J Clin Invest 113:981-9; Hammoud et al., 2005 J Neurovirol 11:346-55; Wiley et al., 2006 J Neurovirol 12:262-71), amyotrophic lateral sclerosis (Turner et al., 2004 Neurobiol Dis 15:601-9), corticobasal degeneration (Henkel et al., 2004 Mov Disord 19:817-21; Gerhard et al., 2004 Mov Disord 19:1221-6), Huntington's disease (Pavese et al., 2006 Neurology 66:1638-43), Cancer (Hardwick et al., 2002 Cancer Genet Cytogenet. 139(1):48-51; Papadopoulo V. 2003 Ann Pharm Fr. 61(1):30-50; Han Z., 2003 J Recept Signal Transduct Res. 23(2-3):225-38), Alzheimer's disease (Papadopoulo V. 2003 Ann Pharm Fr. 61(1):30-50; Li et al., 2007 Biochem Pharmaco. 73(4):491-503), depression (Gavioli E C., 2003 Eur J Pharmacol. 13;471(1):21-6; Kita A. 2004 Br J Pharmacol. 142(7):1059-72) and Cancer, auto-immune, infectious and neurodegenerative diseases (Galiegue et al., 2003 Curr Med Chem 10: 1563-72). It is widely acknowledged that ligands of the TSPO may be of benefit in the treatment of such diseases.

The TSPO is densely distributed in most peripheral organs including the lungs, heart and kidneys, yet it is only minimally expressed in the normal brain parenchyma. Following neuronal injury or infection, TSPO expression in the brain parenchyma is dramatically increased. In vitro autoradiography and immunohistochemistry has revealed that elevated TSPO binding in this region directly correlated with the appearance of activated microglia. Recently, in vivo positron emission tomography (PET) imaging in patients suffering from Alzheimer's disease (AD) and multiple sclerosis (MS) confirmed that TSPO binding in the brain parenchyma was confined to activated microglial cells.

Microglia are the principal immune effector cells of the central nervous system (CNS). These macrophage-like immune cells are assumed to derive from monocytic lineage and their primary role lies in host defense and immune surveillance. They are highly sensitive to changes in their microenvironment and rapidly become activated in response to pathological events. For this reason, the TSPO is believed to be intimately associated with initial inflammatory processes in the early stages of several neurodegenerative disorders.

A number of classes of TSPO ligands have been reported over the past few decades including the benzodiazepines (diazepam and Ro 5-4864), isoquinoline carboxamides (PK 11195), indoleacetamides (FGIN-1-27), phenoxyphenyl-acetamides (DAA1106), pyrazolopyrimides (DPA-713), benzothiazepines and imidazopyridines. Some other classes have also been developed. However, a more extensive range of ligands with varying binding properties and biological activity is required to better characterise the physiological and therapeutic roles of TSPO, its exact localisation and the anticipated existence of TSPO subtypes.

The isoquinoline carboxamide [¹¹C](R)-PK 11195 has been used as a pharmacological probe for studying the function and expression of TSPO. A number of PET studies conducted in patients with AD, MS and multiple system atrophy (MSA) has shown that measurement of TSPO in vivo with [¹¹C](R)-PK 11195 is feasible in the living brain. Although [¹¹C](R)-PK 11195 is regarded as the most widely used PET TSPO ligand it displays a poor signal to noise ratio and has demonstrated low brain permeability which ultimately decreases its sensitivity as a marker of microglial activation.

In 1998, the phenoxyphenyl-acetamide derivative, DAA1106, was reported as a highly selective and potent ligand for the TSPO (Chaki, S.; Funakoshi, T.; Yoshikawa, R.; Okuyama, S.; Okubo, T.; Nakazato, A.; Nagamine, M.; Tomisawa, K. European Journal of Pharmacology, 1999, 371, 197-204). Recently, DAA1106 was labelled with carbon-11 (¹¹C) and used in PET studies to evaluate its in vivo kinetics in both rodent and primate brains (Zhang M R, Kida T, Noguchi J et al. [¹¹C]DAA1106: radiosynthesis and in vivo binding to peripheral benzodiazepine receptors in mouse brain. Nucl Med Biol 2003; 30:513-519. Maeda J, Suhara T, Zhang M R et al. Novel peripheral benzodiazepine receptor ligand [¹¹C]DAA1106 for PET: An imaging tool for glial cells in the brain. Synapse. 2004;52:283-291). The binding of [¹¹C]DAA1106 was shown to be four times greater than [¹¹C](R)-PK 11195 in the monkey occipital cortex, indicating its superior brain permeability. A fluorine-18 (¹⁸F) analogue of this compound has also been synthesised, namely [¹⁸F]FEDAA1106, and this analogue also displays similar binding characteristics in vivo to [¹¹C]DAA1106 (Zhang M R, Maeda J, Ogawa M et al. J Med Chem. 2004;47:2228-2235. The binding of both [¹¹C]DAA1106 and [¹⁸F]FEDAA1106, however, appear to be irreversible and, in fact, their slow elimination from the brain indicates that they may not have suitable kinetics for quantitative analysis.

Ryu J K et al, Neurobiology of Disease, 20 (2005) 550-561 reports that the TSPO ligand PK 11195 reduces microglial activation and neuronal death in quinolinic acid-injected rat stratum. The results reported in this paper suggest that inflammatory responses from activated microglia are damaging to striatal neurons and thus pharmacological targeting of TSPO in microglia is likely to protect neurons in neurological disorders.

In published international application WO 2008/022396, it was also generally discloses that certain imidazopyridazines labelled with ¹⁸F show radioactivity uptake in tissue rich in PBR.

It would be advantageous to identify TSPO ligands with improved brain kinetics that can be used to image TSPO expression in vivo, as such ligands could be utilised to further study the cascade of biochemical events involved in the initial stages of several neurodegenerative disorders. It would also be advantageous to identify TSPO ligands with improved brain kinetics as such ligands have potential to serve as both diagnostic and therapeutic tools for neurodegenerative disorders.

It is an object of the present invention to overcome or ameliorate at least one of the disadvantages of the prior art, or to provide a useful alternative.

It is an object of the invention in a preferred form to provide compounds that bind TSPO, processes for their preparation and methods for their use. Specifically, it is an object of the invention in a preferred form to provide compounds and methods for imaging translocator protein TSPO expression in a subject. It is also an object of the invention in a preferred form to provide compounds and methods for the treatment of disorders, in particular neurodegenerative disorders, inflammation or anxiety.

### SUMMARY OF THE INVENTION

According to a first aspect, the present invention provides, a compound of formula (I)

X-L-Y (I)

wherein,

X and Y independently bind TSPO, wherein X and Y are the same or different; and

L is a linker that links X to Y;

or a salt or solvate thereof.

Preferably, X and Y are independently selected from

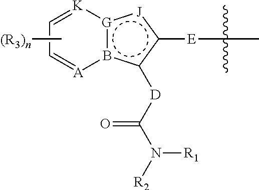

wherein,

A and K are independently CH, C or N, J is CH or N, and B and G are independently C or N provided that at least one of B and G is C, wherein at least two of A, B, G, J and K are N; D is O, NH, (CH₂)ₘ or S; E is an aryl group or a heteroaryl group optionally substituted with one or more of the following substituents: halogen, C₁-C₁₀ alkyl, C₂-C₁₀ alkenyl, C₂-C₁₀ alkynyl, TC₁-C₆ alkyl, TC₂-C₁₀ alkenyl, or TC₂-C₁₀ alkynyl, each of which is optionally substituted with one or more halogen substituents, and wherein T is NH, O or S; R₁ and R₂ are independently hydrogen, C₁-C₁₀ alkyl, C₂-C₁₀ alkenyl, C₂-C₁₀ alkynyl, aryl or heteroaryl, each being optionally substituted with one of more halogen;

or R₁ and R₂ together with the nitrogen to which they are attached, form a heterocylic ring having between 3 and 7 ring members, optionally substituted with one of more halogen; R₃ is independently halogen, C₁-C₁₀ alkyl, C₂-C₁₀ alkenyl, C₂-C₁₀ alkynyl, TC₁-C₆ alkyl, TC₂-C₁₀ alkenyl or TC₂-C₁₀ alkynyl, each of which is optionally substituted with one or more halogen substituents, and wherein T is NH, O or S;

m is a number between 1 and 6; and

n is a number between 0 and 3.

In one embodiment, A, G and J are N, K is CH or C and B is C; or A, B and J are N, K is CH or C and G is C. Preferably, R₃ is a C₁-C₆ alkyl, and wherein n is 1 or 2. More preferably, n is 2 and each respective R₃ is methyl. In a preferred embodiment, respective methyl groups are positioned meta to each other.

Preferably, D is (CH₂)ₘ, and wherein m is 1. In further embodiments, R₁ and R₂ are independently a C₁-C₆ alkyl. In alternative embodiments, R₁ and R₂ are independently ethyl. In further embodiments, E is a 5-, or 6-membered aryl or heteroaryl group optionally substituted with one or more of the following substituents: halogen, C₁-C₆ alkyl, C₂-C₆ alkenyl, and C₂-C₆ alkynyl. In a preferred embodiment, E is phenyl.

In a particularly preferred embodiment, X and Y are independently

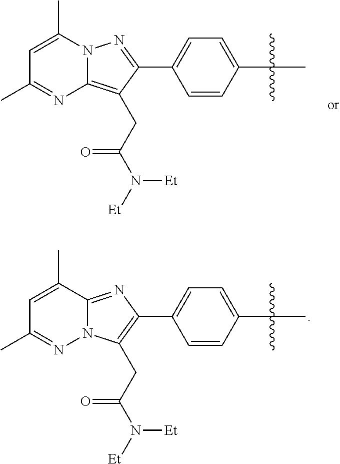

In certain embodiments, L is preferably selected from the group consisting of C₁-C₂₀ alkyl, C₂-C₂₀ alkenyl, C₂-C₂₀ alkynyl, T(_{C}₁-C₂₀ alkyl)T, T(C₂-C₂₀ alkenyl)T, T(C₂-C₂₀ alkynyl)T, TCH₂(CH₂OCH₂)ₚCH₂T; TCH₂(CH₂NHCH₂)ₚCH₂T, amino acids including but not limited to glycine oligimers; wherein T is NH, O or S; and wherein p is a number between 1 and 10.

In preferred embodiments, L is selected from the group consisting of O(C₁-C₂₀ alkyl)O, O(C₂-C₂₀ alkenyl)O, O(C₂-C₂₀ alkynyl)O and OCH₂(CH₂OCH₂)ₚCH₂O; wherein p is a number between 1 and 10.

A compound of formula (I) is preferably selected from the group consisting of:

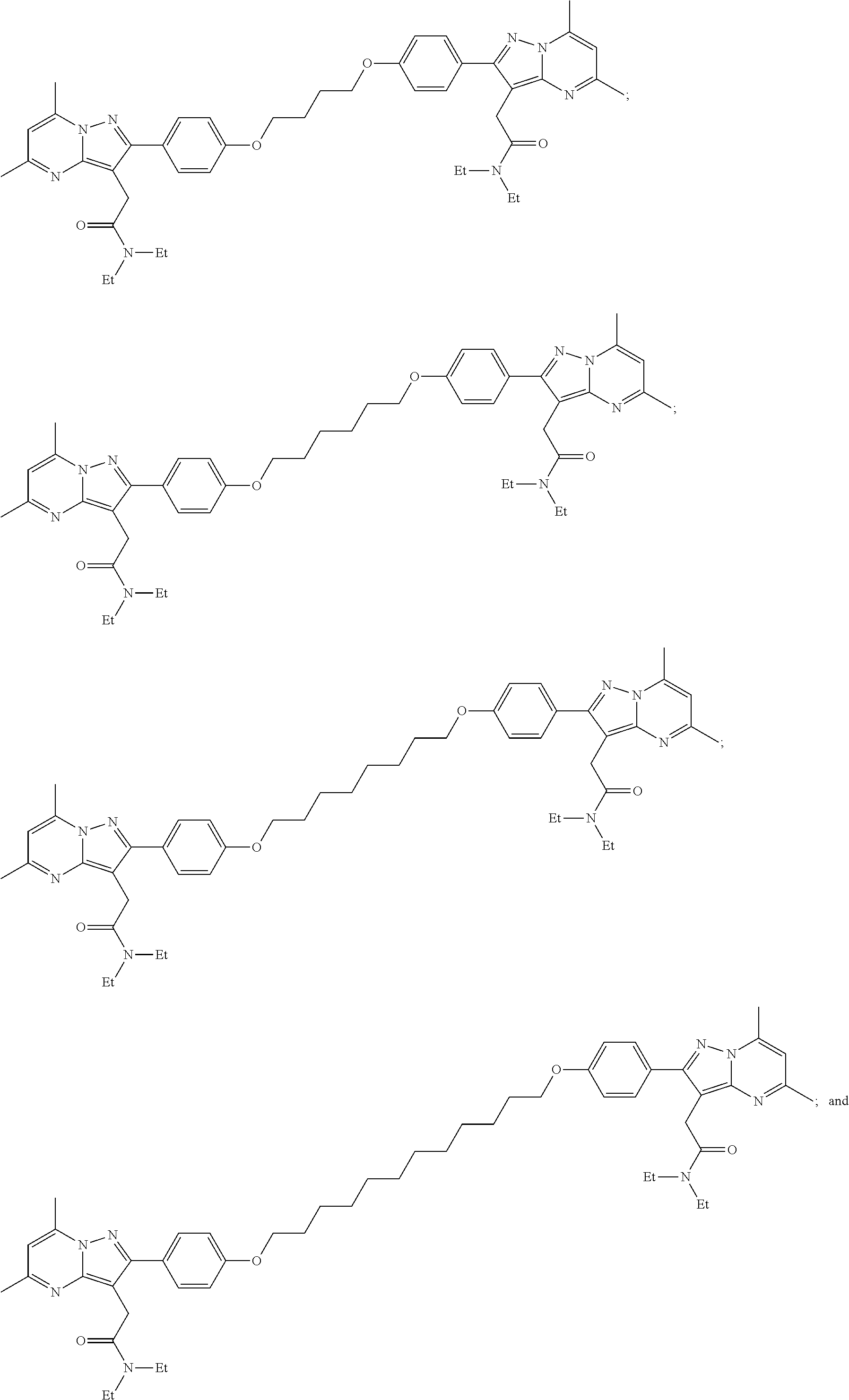

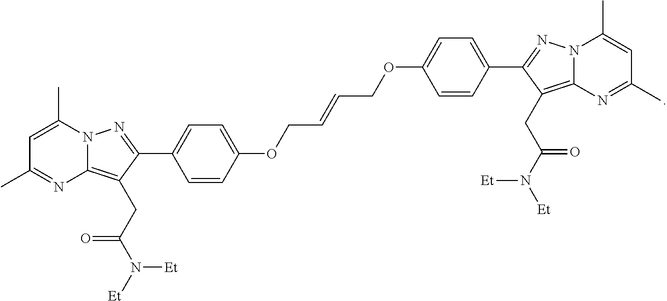

Preferably, a compound of formula (I) selected from the group consisting of:

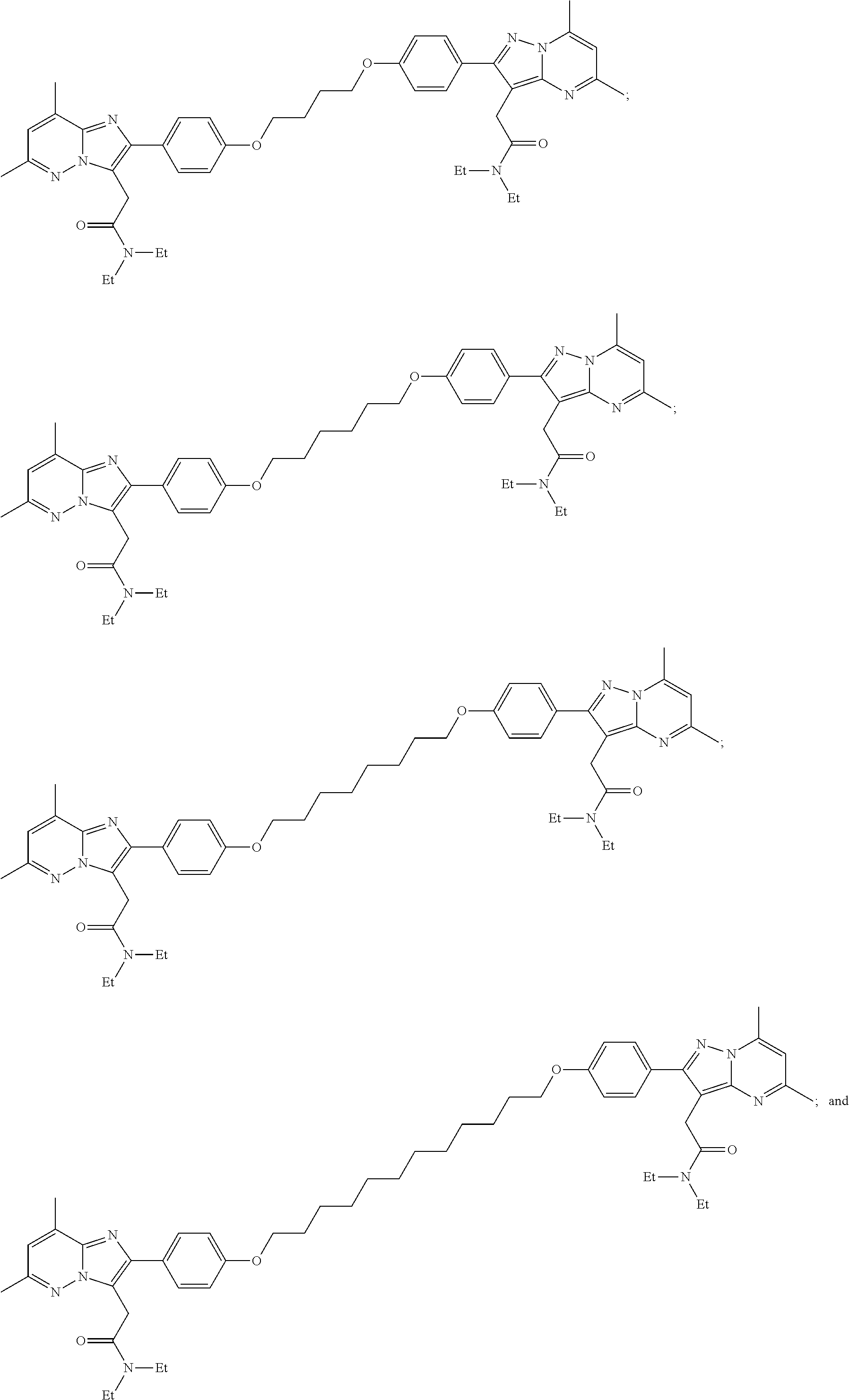

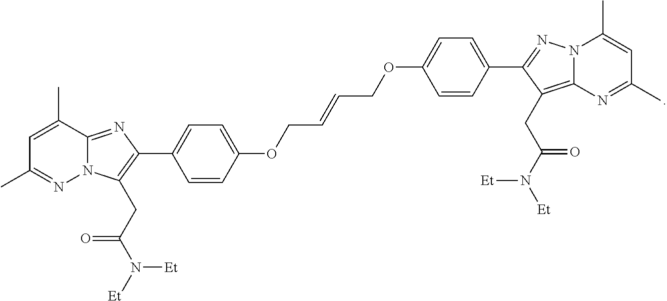

In a preferred embodiment, the compound of formula (I) according to the first aspect is radiolabelled with a radioisotope. Preferably, the radioisotope is selected from the group consisting of ¹⁸F, ¹²³I, ⁷⁶Br, ¹²⁴I and ⁷⁵Br. Preferably, the radioisotope is ¹⁸F.

According to a second aspect the present invention provides a pharmaceutical composition comprising a compound according to the first aspect or a pharmaceutically acceptable salt thereof, and a pharmaceutically acceptable carrier.

According to a third aspect, the present invention provides a method of diagnosing a disorder in a subject, comprising administering to a subject a compound of formula (I) according to the first. Preferably, the method comprises imaging translocator protein (1 8 kDa) (TSPO) in the subject. In one embodiment, when the compound is radiolabelled with a radioisotope, the radioisotope is selected from the group consisting of ¹⁸F, ¹²³I, ¹²⁴I, ⁷⁵Br and ⁷⁶Br. In a preferred embodiment, the method comprises obtaining an image indicating the location of the protein. In a more preferred embodiment, the image is obtained by positron emission tomography (PET) imaging. Preferably, the compound of formula (I) is radiolabelled with ¹²³I and the image is obtained by SPECT imaging. In one embodiment, the image is obtained to assess the extent of TSPO binding of the compound or salt thereof in the brain parenchyma of the subject. Preferably, the disorder is a neurodegenerative disorder, inflammation or anxiety. Preferably, the disorder is selected from the group consisting of: Alzheimer's disease, Parkinson's disease, Huntington's disease, multiple sclerosis, multiple system atrophy, epilepsy, encephalopathy, stroke, brain tumour, anxiety, stress, emotional disturbances or cognitive impairment, glioblastoma, ischemic stroke, herpes encephalitis, HIV, amyotrophic lateral sclerosis, corticobasal degeneration, cancer, depression, an auto-immune disease and an infectious disease. Preferably, the subject is a human.

According to a fourth aspect, the present invention provides use of a compound according to the first aspect in the manufacture of an agent for diagnosing a disorder in a subject. Preferably, diagnosing the disorder comprises imaging translocator protein (18 kDa) in the subject. More preferably, the compound of formula (I) is radiolabelled with ¹²³I a translocator protein image is obtained by SPECT imaging. In one embodiment, the disorder is a neurodegenerative disorder, inflammation or anxiety. In a preferred embodiment, the disorder is selected from the group consisting of: Alzheimer's disease, Parkinson's disease, Huntington's disease, multiple sclerosis, multiple system atrophy, epilepsy, encephalopathy, stroke, brain tumour, anxiety, stress, emotional disturbances or cognitive impairment, glioblastoma, ischemic stroke, herpes encephalitis, HIV, amyotrophic lateral sclerosis, corticobasal degeneration, cancer, depression, an auto-immune disease and an infectious disease.

According to a fifth aspect, the present invention provides use of a compound of the first aspect in the manufacture of a medicament for the treatment of a disorder in a subject. Preferably, the disorder is characterised by an abnormal density of TSPO receptors in a mammal. In one embodiment, the disorder is a neurodegenerative disorder, inflammation or anxiety. In a preferred embodiment, the disorder is selected from the group consisting of: Alzheimer's disease, Parkinson's disease, Huntington's disease, multiple sclerosis, multiple system atrophy, epilepsy, encephalopathy, stroke, brain tumour, anxiety, stress, emotional disturbances or cognitive impairment, glioblastoma, ischemic stroke, herpes encephalitis, HIV, amyotrophic lateral sclerosis, corticobasal degeneration, cancer, depression, an auto-immune disease and an infectious disease.

According to a sixth aspect, the present invention provides a method for treating a disorder in a subject comprising administering to the subject a compound according to the first aspect. In a preferred embodiment, the disorder is characterised by an abnormal density of TSPO receptors in a mammal. More preferably, the disorder is a neurodegenerative disorder, inflammation or anxiety in a subject. In a most preferred embodiment, the disorder is Alzheimer's disease, Parkinson's disease, Huntington's disease, multiple sclerosis, multiple system atrophy, epilepsy, encephalopathy, stroke, brain tumour, anxiety, stress, emotional disturbances or cognitive impairment, glioblastoma, ischemic stroke, herpes encephalitis, HIV, amyotrophic lateral sclerosis, corticobasal degeneration, cancer, depression, auto-immune and infectious diseases. According to a third aspect, the present invention provides a method of diagnosing a disorder in a subject, comprising administering to a subject a compound of formula (I) as defined in the first aspect. Preferably, the method comprises imaging translocator protein (18 kDa) (TSPO) in the subject.

According to a seventh aspect, the present invention provides a process for preparing a compound of formula (I), said process comprising reacting a compound of formula (II) with V-L-V in the Presence of a base

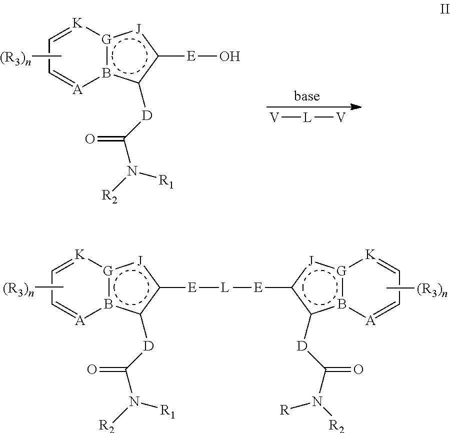

wherein,
A and K are independently CH, C or N, J is CH or N, and B and G are independently C or N provided that at least one of B and G is C, wherein at least two of A, B, G, J and K are N;
D is O, NH, (CH₂)ₘ or S;
E is an aryl group or a heteroaryl group optionally substituted with one or more of the following substituents: halogen, C₁-C₁₀ alkyl, C₂-C₁₀ alkenyl, C₂-C₁₀ alkynyl, TC₁-C₆ alkyl, TC₂-C₁₀ alkenyl, or TC₂-C₁₀ alkynyl, each of which is optionally substituted with one or more halogen substituents, and wherein T is NH, O or S;
R₁ and R₂ are independently hydrogen, C₁-C₁₀ alkyl, C₂-C₁₀ alkenyl, C₂-C₁₀ alkynyl, aryl or heteroaryl, each being optionally substituted with one of more halogen;
or R₁ and R₂ together with the nitrogen to which they are attached, form a heterocylic ring having between 3 and 7 ring members, optionally substituted with one of more halogen;
R₃ is independently halogen, C₁-C₁₀ alkyl, C₂-C₁₀ alkenyl, C₂-C₁₀ alkynyl, TC₁-C₆ alkyl, TC₂-C₁₀ alkenyl or TC₂-C₁₀ alkynyl, each of which is optionally substituted with one or more halogen substituents, and wherein T is NH, O or S;
m is a number between 1 and 6; and
n is a number between 0 and 3;

L is selected from the group consisting of C₁-C₂₀ alkyl, C₂-C₂₀ alkenyl, C₂-C₂₀ alkynyl, T(C₁-C₂₀ alkyl)T, T(C₂-C₂₀ alkenyl)T, T(C₂-C₂₀ alkynyl)T, TCH₂(CH₂OCH₂)ₚCH₂T; TCH₂(CH₂NHCH₂)ₚCH₂T, amino acids including but not limited to glycine oligimers;

wherein T is NH, O or S;

wherein p is a number between 1 and 10;

wherein V is a leaving group that reacts with a base; and

wherein the base is NaH or K₂CO₃.

According to a eighth aspect, the present invention provides a compound of formula (I) according to the first aspect capable of eliciting a response when bound to a TSPO receptor.

Without wishing to be bound by theory, X and Y independently bind TSPO though interaction with two sites in the same protein or by binding across two separate proteins. Preferably each one of X and Y independently binds TSPO, however, it will be appreciated that under select conditions, only one of X or Y may bind with the TSPO receptor at any one time. It will also be appreciated that the nature and type of binding of the compounds of formula (I) to TSPO will be dependent on X and Y and the length of the linker L.

The linker L may be any suitable linker capable of connecting X to Y. Suitable linkers include although are not limited to covalent bonds, organic chains, inorganic chains, organometallic chains, polymers and the like. The linker may also be a single atom or simple functional group. The linker may also include an amino acid, including but not limited to glycine oligimers. Suitable glycine oligimers include oligoglycol units attached to a methylenediacyl core, for example

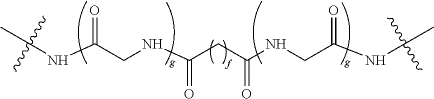

wherein

g is a number between 1 and 4; and

f is a number is a number between 1 and 4.

It will be appreciated that each g is independently 1, 2, 3 or 4, and f is 1, 2, 3 or 4.

Preferably X and Y are derived from compounds, which as independent units absent the linker L, elicit a response when bound to the TSPO.

In the structure

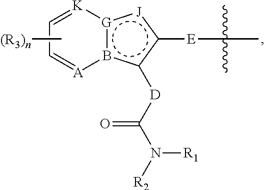

the symbol

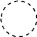

represents a degree of unsaturation around the five membered ring to which it is associated. It will be appreciated that when J is CH, and B and G are independently selected from the group consisting of C and N provided that at least one of B and G is C, the five membered ring to which J is attached will be non-aromatic, as represent by

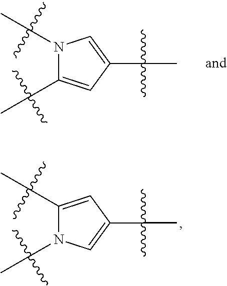

whereas when J is N, it will be appreciated that the ring is aromatic as represented by

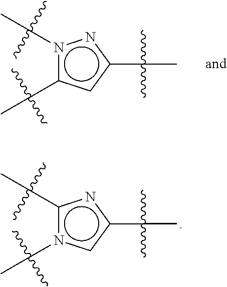

It will be appreciated that when A and/or K is C, R₃ is bound to C.

When X and Y are independently selected from

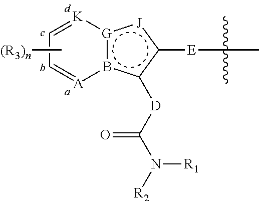

and wherein when n is greater than 0, it will be appreciated that R₃ can be located at any one of the positions a, b, c or d. For example, when n is 1, R₃ is bound at positions a, b, c or d; when n is 2, R₃ is bound at positions a and b, a and c, a and d, b and c, b and d or c and d; when n is 3, R₃ is bound at positions a, b and c; a, b and d; a, c and d; or b, c and d; when n is 4, R₃ is bound at positions a, b, c and d. Preferably R₃ is bound at positions b and d. More preferably n is 2 and R₃ is bound at positions a and c or b and d. i.e. each R₃ is attached to the ring at positions meta to each other.

### BRIEF DESCRIPTION OF THE DRAWING

FIG. 1 shows dose-response curves depicting the dose-dependent displacement of [³H]PK11195 binding in HEK293 cells transfected with human TSPO, in the presence of various bidentate ligands at concentrations ranging from 0.01 nM to 1 μM. Binding data is fit to one of two curves; one-site competition versus two-site competition.

### DETAILED DESCRIPTION OF THE INVENTION

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

As used herein, the term “alkyl” refers to a straight chain, branched or mono- or poly-cyclic alkyl. Typically, the alkyl is a C₁ to C₂₀ alkyl, for example, an alkyl group having from 1 to 20 carbon atoms e.g. 1, 2, 3, 4, 5, 6, 7, 8, 9, 10, 11, 12, 13, 14, 15, 16, 17, 18, 19 or 20 carbon atoms. The alkyl group may have from 1 to 2, 1 to 4, 1 to 6, 1 to 8, 1 to 10, 1 to 12, 1 to 14, 1 to 16, 1 to 18 or 1 to 20 carbon atoms.

Examples of straight chain and branched alkyl include but are not limited to methyl, ethyl, propyl, isopropyl, butyl, isobutyl, sec-butyl, tert-butyl, pentyl, isopentyl, sec-pentyl, 1,2-dimethylpropyl, 1,1-dimethylpropyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, heptyl, octyl, nonyl, decyl, undecyl, dodecyl, tridecyl, tetradecyl, pentadecyl, hexadecyl, heptadecyl, octadecyl, nonadecyl and icosyl.

Examples of cyclic alkyl include cyclopropyl, cyclobutyl, cyclopentyl and cyclohexyl.

As used herein, the term “alkenyl” refers to a straight chain, branched or cyclic alkenyl. Typically, the alkenyl is a C₂ to C₂₀ alkenyl, for example, an alkenyl group having from 2 to 20 carbon atoms e.g. 2, 3, 4, 5, 6, 7, 8, 9, 10, 11,12, 13,14, 15,16, 17, 18, 19 or 20 carbon atoms. The alkenyl group may have from 2 to 4, 2 to 6, 2 to 8, 2 to 10, 2 to 12, 2 to 14, 2 to 16, 2 to 18 or 2 to 20 carbon atoms. Preferably the alkenyl group is a C₂ to C₈ alkenyl. Examples of alkenyl include vinyl, allyl, 1-methylvinyl, butenyl, isobutenyl, 3-methyl-2-butenyl, 1-pentenyl, cyclopentenyl, 1-methylcyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1,4-pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl and 1,3,5,7-cyclooctatetraenyl.

It will be appreciated that the C₂ to C₂₀ akenyl may contain between 1 and 10 alkene bonds e.g. 1, 2, 3, 4, 5, 6, 7, 8, 9 or 10 alkene bonds. Each alkene bond may be located at any position in the straight, branched or cyclic chain.

As used herein, the term “alkynyl” refers to a straight chain, branched or cyclic alkynyl. Typically, the alkynyl is a C₂ to C₂₀ alkynyl for example, an alkynyl group having from 2 to 20 carbon atoms e.g. 2, 3, 4, 5, 6, 7, 8, 9, 10, 11, 12, 13, 14, 15, 16, 17, 18, 19 or 20 carbon atoms. The alkynyl group may have from 2 to 4, 2 to 6, 2 to 8, 2 to 10, 2 to 12, 2 to 14, 2 to 16, 2 to 18 or 2 to 20 carbon atoms. Preferably the alkynyl group is a C₂ to C₆ alkynyl.

It will be appreciated that the C₂ to C₂₀ akynyl may contain between 1 and 10 alkyne bonds e.g. 1, 2, 3, 4, 5, 6, 7, 8, 9 or 10 alkyne bonds. Each alkyne bond may be located at any position in the straight, branched or cyclic chain.

As used herein, the term “aryl” refers to a radical of a single, polynuclear, conjugated or fused aromatic hydrocarbon or aromatic heterocyclic ring system. Preferably the aryl group has from 4 to 20 carbon atoms. e.g. 4, 5, 6, 7, 8, 9, 10, 11, 12, 13, 14, 15, 16, 17, 18, 19, or 20 carbon atoms. The aryl group may have from 4 to 6, 4 to 8, 4 to 10, 4 to 12, 4 to 14, 4 to 16 or 4 to 18 carbon atoms. Preferably the aryl group has 6 to 8, 6 to 10, 6 to 12, 6 to 14, 6 to 16, or 6 to 18 carbon atoms. More preferably, the aryl group, has 5 carbon atoms. Even more preferably, the aryl has 6 carbon atoms. Examples of aryl include, although are not limited to phenyl, biphenyl, naphthyl, tetrahydronaphthyl, indenyl, azulenyl, phenantryl, pyrenyl and the like. Any available position of the aromatic residue can be used for attachment to the remainder of the molecule of formula (I).

As used herein, the term “heteroaryl” refers to single, polynuclear, conjugated and fused aromatic radical having preferably between 5 and 20 ring atoms, wherein 1 to 6, or 1 to 5, or 1 to 4, or 1 to 3, or 1 or 2 of these ring atoms are heteroatoms independently variable and independently selected from the group consisting of: N, NH, O and S. The heteroaryl group may have from 4 to 10, 4 to 12, 4 to 14, 4 to 16, 4 to 18, 4 to 19, 6 to 10, 6 to 12, 6 to 14, 6 to 16, 6 to 18 or 6 to 19 carbon atoms. The heteroaryl group may have 1 to 2, 1 to 3, 1 to 4, 1 to 5 or 1 to 6 heteroatoms. The hetero atoms may be independently selected from the group consisting of: N and NH, N and O, NH and O, N and S, NH and S and S and O. Examples of such heteroaryl groups include but are not limited to pyridyl, thienyl, furyl, pyrryl, indolyl, pyridazinyl, pyrazolyl, pyrazinyl, thiazolyl, pyrimidinyl, quinolinyl, isoquinolinyl, benzofuranyl, benzothienyl, purinyl, quinazolinyl, phenazinyl, acridinyl, benzoxazolyl, benzothiazolyl and the like. Any available position of the heteroaromatic residue can be used for attachment to the remainder of the molecule of formula (I). Nitrogen-containing heteroaryl groups may be substituted at nitrogen with an oxygen atom to form an N-oxide. Sulfur-containing heteroaryl groups may be substituted at sulfur with one or two oxygen atoms to form a sulfoxide or a sulfone respectively.

As used herein, the term “halo” and “halogen” refer to a halogen radical, e.g. fluoro, chloro, bromo or iodo.

As used herein, a reference to a group “optionally substituted” means the group may be substituted with one or more substituents. For example, in certain embodiments a group may be optionally substituted with one or more halogen radicals.

Acronyms used throughout the specification have the following meanings:
AD=Alzheimer's disease
ANC=Adenine nucleotide carrier
CBR=central benzodiazepine receptor
CNS=central nervous system
MPTP=mitochondrial permeability transition pore
MS=multiple sclerosis
PBR=Peripheral benzodiazepine receptor
PET=Positron emission tomography
SPECT=single photon emission computed tomography
TSPO=Translocator protein (18 kDa)
VDAC=voltage-dependent anion channel

The compounds of formula (I) can be used to bind TSPO. In particular, when radiolabelled with a radioisotope, the compounds can be used as accurate in vivo markers of TSPO and therefore microglial activation. These compounds can therefore be used to study neuropathological events in a number of disorders, in particular neurodegenerative disorders. They can be used as a tool for diagnosis of such disorders and for monitoring the progression of the disorders.

The radioisotope can be selected from any suitable radioisotope known to the skilled addressee and include for example radioisotopes listed in the Handbook of Radiopharmaceuticals, Radiochemistry Applications, ed. Michael Welsch and Carol S. Redvanly, John Wiley & Sons Ltd 2003; and PET Chemistry, The Driving Force for Molecular Imaging. Ed. P. A. Schubiger, L. Lehmann, M. Friebe, Springer 2007. Useful radioisotopes include, although are not limited to, ¹⁸F, ¹²³I, ⁷⁶Br, ¹²⁴I and ⁷⁵Br and ¹¹C.

As used herein, by a compound of formula (I), “radiolabelled” with ¹⁸F, ¹²³I, ⁷⁶Br, ¹²⁴I and ⁷⁵Br, it is meant that at least one substituent on the compound has a radiolabel isotope of ¹⁸F, ¹²³I, ⁷⁶Br, ¹²⁴I and ⁷⁵Br present.

For example, in the compound of formula (I), any one or more of the following substituents X, Z or L may be radiolabelled with ¹⁸F, ¹²³I, ⁷⁶Br, ¹²⁴I or ⁷⁵Br.

X-L-Y (I)

wherein,
X and Y independently bind TSPO, wherein X and Y are the same or different;

and
L is a linker that links X to Y;

radiolabelled with a radiolabel isotope or a salt or solvate thereof.

Typically, when the compound of formula (I) is radiolabelled with ¹⁸F, ⁷⁶Br, ¹²⁴I and or ⁷⁵Br, the image is obtained by positron emission tomography (PET) imaging. Typically, when the compound of formula (I) is radiolabelled with ¹²³I, the image is obtained by single positron emission computer tomography (SPECT) imaging.

A number of classes of TSPO ligands have been described in the literature. A compound which is effective as a therapeutic drug is not necessarily a compound that can be radiolabelled and used for imaging. Indeed, many drugs that are used therapeutically are not selective for a specific target and may interact with several targets to produce a therapeutic effect. Further, many therapeutic drugs do not have affinity that is in the nM range normally used for imaging, but have affinity in the μM range. In addition, the metabolism and lipophilicity of a therapeutic drug, particularly when administered at tracer levels for imaging, may make the drug unsuitable for use for imaging. The compounds of formula (I) radiolabelled with a radioisotope selected from ¹⁸F, ¹²³I, ⁷⁶Br, ¹²⁴I and ⁷⁵Br can be used to image TSPO and therefore microglial activation in a subject.

The compounds of formula (I) radiolabelled with a radioisotope selected from ¹⁸F, ¹²³I, ⁷⁶Br, ¹²⁴I and ⁷⁵Br form salts, and salts of such compounds are encompassed by the present invention. The salts are preferably pharmaceutically acceptable, but it will be appreciated that non-pharmaceutically acceptable salts also fall within the scope of the present invention. Examples of pharmaceutically acceptable salts include salts of pharmaceutically acceptable cations such as sodium, potassium, lithium, calcium, magnesium, ammonium and alkylammonium; acid addition salts of pharmaceutically acceptable inorganic acids such as hydrochloric, orthophosphoric, sulphuric, phosphoric, nitric, carbonic, boric, sulfamic and hydrobromic acids; or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulphonic, trihalomethanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

Compounds of formula (I) can be radiolabelled with ¹⁸F, ¹²³I, ⁷⁶Br, ¹²⁴I or ⁷⁵Br by standard techniques known in organic chemistry for modifying an organic compound to replace a hydrogen or halo group in the compound with ¹⁸F, ¹²³I, ⁷⁶Br, ¹²⁴I or ⁷⁵Br. (VICTOR WILLIAM PIKE. THE STATUS OF PET RADIOCHEMISTRY FOR DRUG DEVELOPMENT AND EVALUATION. Drug Information Journal, Vol. 31, pp. 997-1013, 1997).

Alternatively, compounds of formula (I) radiolabelled with a radioisotope selected from ¹⁸F, ¹²³I, ⁷⁶Br, ¹²⁴I and ⁷⁵Br may be prepared by incorporating ¹⁸F, ¹²³I, ⁷⁶Br, ¹²⁴I or ⁷⁵Br as a substituent in one of the starting materials or in an intermediate used in the synthesis of compounds of formula (I).

A compound of formula (I) radiolabelled with ¹⁸F, ¹²³I, ⁷⁶Br, ¹²⁴I or ⁷⁵Br may, for example, be prepared by preparing a compound having the formula (I) defined above, but with a leaving group, such as tosylate, mesylate, Br or I, that allows an aliphatic nucleophilic substitution reaction to occur at the leaving group, and then subjecting the compound to conditions under which an aliphatic nucleophilic substitution reaction occurs to replace the leaving group with ¹⁸F, ¹²³I, ⁷⁶Br, ¹²⁴I or ⁷⁵Br. For example, when the leaving group is Br or tosylate, the compound may be reacted with the [¹⁸F]-kryptofix-K222 complex in acetonitrite at about 80° C. for 10 minutes to form a compound of formula (I) radiolabelled with ¹⁸F. Compounds of formula (I) radiolabelled with ¹²³I, ⁷⁶Br, ¹²⁴I or ⁷⁵Br may also be formed by forming a compound having the formula (I) defined above, but with a stannyl, silyl or halogen (the halogen substituent is usually different to the radioisotope), and subjecting the compound to an electrophilic substitution reaction in acetic media using an oxidising agent such as chloramine-T to form a compound of formula (I) radiolabelled with ¹²³I, ⁷⁶Br, ¹²⁴I or ⁷⁵Br. In some embodiments, this reaction may be carried out at room temperature, and in other embodiments, the reaction mixture is heated to about 80° C. to 100° C. A compound of formula (I) as defined above, substituted with a leaving group may be modified by reactions known in organic chemistry to introduce a leaving group as a substituent anywhere on the compound.

The compounds of formula (I) may be radiolabelled with ¹⁸F (half-life 110 minutes), ¹²³I (half-life 13.2 hours), ⁷⁶Br (half-life 16.2 hours), ¹²⁴I (half-life 4.2 days) or ⁷⁵Br (half-life 1.6 hours). Typically, the compounds of formula (I) are radiolabelled with ¹⁸F. Compounds of formula (I) radiolabelled with ¹⁸F, ¹²³I, ⁷⁶Br, ¹²⁴I or ⁷⁵Br are more practical in a clinical sense for imaging than compounds radiolabelled with radioisotopes having a significantly shorter half-life, as multiple scans can be performed on one day. In addition, hospitals/organisations that do not have a cyclotron on site can use such radioligands, as the radioligands can be prepared offsite and transported to the hospital/organisation with no significant loss of activity during transportation. In addition, longer scans (e.g. 180 minutes) can be undertaken with compounds labelled with ¹⁸F, ¹²³I, ⁷⁶Br, ¹²⁴I or ⁷⁵Br making them more appropriate for the study of most biological processes.

Compounds of formula (I) radiolabelled with ¹⁸F, ¹²³I, ⁷⁶Br, ¹²⁴I or ⁷⁵Br may have high affinity and selectivity for TSPO, and may be used for imaging TSPO in a subject. Accordingly, compounds of formula (I) radiolabelled with ¹⁸F, ¹²³I, ⁷⁶Br, ¹²⁴I or ⁷⁵Br may be used to study TSPO in a subject.

In a subject having a neurodegenerative disorder, TSPO expression in the brain parenchyma is dramatically increased compared to a subject not having a neurodegenerative disorder. Accordingly, the compounds of formula (I) radiolabelled with ¹⁸F, ¹²³I, ⁷⁶Br, ¹²⁴I or ⁷⁵Br may be used to study neurodegenerative disorders and may be used to diagnose and monitor the progression of neurodegenerative disorders. Neurodegenerative disorders that can be studied, diagnosed or monitored using these compounds include Alzheimer's disease, multiple sclerosis, Parkinson's disease, Huntington's disease, multiple system atrophy, epilepsy, encephalopathy, stroke and brain tumours. Each of these disorders is associated with neuronal injury or infection. Other disorders that may be studied, diagnosed or monitored using these compounds include anxiety, stress, emotional disturbances or cognitive impairment, glioblastoma, multiple sclerosis, ischemic stroke, herpes encephalitis, Parkinson's disease, HIV, amyotrophic lateral sclerosis, corticobasal degeneration, Huntington's disease, Cancer, depression, auto-immune and infectious diseases.

In accordance with the present invention, a compound of formula (I) radiolabelled with a radioisotope selected from ¹⁸F, ¹²³I, ⁷⁶Br, ¹²⁴I and ⁷⁵Br or a pharmaceutically acceptable salt thereof is administered to the subject. When the compound of formula (I) is radiolabelled with ¹⁸F, ₇₆Br, ¹²⁴I or ⁷⁵Br, the image of the location of the radioisotope in the subject, and therefore the location of TSPO in the subject, may be obtained by positron emission tomography (PET) imaging using conventional techniques known the art. (R J Hargreaves. The Role of Molecular Imaging in Drug Discovery and Development. Clinical pharmacology & Therapeutics 2008 VOLUME 83 NUMBER 2, 349-352).

When the compound is radiolabelled with ¹²³I, the image of the location of the radioisotope in the subject may be obtained by SPECT imaging using conventional techniques known in the art. Typically for both PET and SPECT imaging, the data is acquired using conventional dynamic or list mode acquisition techniques, commencing immediately after administration of the compound of formula (I) radiolabelled with ¹⁸F, ¹²³I, ⁷⁶Br, ¹²⁴I or ⁷⁵Br or pharmaceutically acceptable salt thereof, and continuing for about 40 minutes or longer. At the completion of data acquisition, the data is typically processed to provide a time-series of 3D reconstructions, each depicting the distribution of the radioisotope in the body at a particular point in time.

Typically, the compounds of formula (I) radiolabelled with ¹⁸F, ¹²³I, ⁷⁶Br, ¹²⁴I or ⁷⁵Br or pharmaceutically acceptable salt thereof is administered parenterally. Typically, the compounds of formula (I) radiolabelled with ¹⁸F, ¹²³I, ⁷⁶Br, ¹²⁴I or ⁷⁵Br or pharmaceutically acceptable salt thereof is administered parenterally by intravenous injection or infusion. Typically the compound of formula (I) radiolabelled with ¹⁸F, ₇₆Br, ¹²⁴I or ⁷⁵Br or pharmaceutically acceptable salt thereof is administered at a dose in the range of about 5 to 20 mCi (185-740 MBq).

Typically, the compounds of formula (I) radiolabelled with ¹⁸F, ¹²³I, ⁷⁶Br, ¹²⁴I or ⁷⁵Br or pharmaceutically acceptable salt thereof is administered by administering a pharmaceutical composition comprising the compound of formula (I) radiolabelled with ¹⁸F, ¹²³I, ⁷⁶Br, ¹²⁴I or ⁷⁵Br, or pharmaceutically acceptable salt thereof, and a pharmaceutically acceptable carrier.

Preparations for parenteral administration are typically in the form of a sterile aqueous or non-aqueous solution, suspension or emulsion. Examples of suitable non-aqueous solvents are propylene glycol, polyethylene glycol, vegetable oils such as olive oil, and injectable organic esters such as ethyl oleate. Suitable aqueous carriers include water and alcoholic/aqueous solutions, emulsions or suspensions, including saline and buffered media. Suitable parenteral vehicles include sodium chloride solution.

The salts of the compound of formula (I) are preferably pharmaceutically acceptable, but it will be appreciated that non-pharmaceutically acceptable salts also fall within the scope of the present invention. Non-pharmaceutically acceptable salts of the compounds of formula (I) may be used as intermediates in the preparation of pharmaceutically acceptable salts of the compounds of formula (I). Examples of pharmaceutically acceptable salts include salts of pharmaceutically acceptable cations such as sodium, potassium, lithium, calcium, magnesium, ammonium and alkylammonium; acid addition salts of pharmaceutically acceptable inorganic acids such as hydrochloric, orthophosphoric, sulphuric, phosphoric, nitric, carbonic, boric, sulfamic and hydrobromic acids; or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulphonic, trihalomethanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

The compounds of formula (I) may be selective for TSPO and may activate TSPO. The activation of TSPO is related to increased synthesis of neurosteroids. The activation of TSPO can therefore increase the concentration of neurosteroids in the brain. These neurosteroids, including progesterone and dehydroepiandrosterone and their metabolites, positively modulate γ-aminobutyric acid (GABA) neurotransmission leading to nonsedative anxiolytic effects which are of therapeutic benefit in memory and stress related disorders. The compounds of formula (I) may also be used as neuroprotective agents for the treatment of neurodegenerative disorders, as anti-inflammatory agents, and as anxiolytic agents.

Accordingly, in another aspect, the present invention provides a method of treating neurodegenerative disorders, inflammation or anxiety in a subject, comprising administering to the subject a therapeutically effective amount of a compound of formula (I) or a pharmaceutically acceptable salt thereof. The disorders that may be treated by the method include Alzheimer's disease, Parkinson's disease, Huntington's disease, multiple sclerosis, multiple system atrophy, epilepsy, encephalopathy, stroke, brain tumour, anxiety, stress, emotional disturbances or cognitive impairment, glioblastoma, ischemic stroke, herpes encephalitis, HIV, amyotrophic lateral sclerosis, corticobasal degeneration, cancer, depression, an auto-immune disease and an infectious disease.

The compounds of formula (I) or pharmaceutically acceptable salt thereof is typically administered by administering a pharmaceutical composition comprising the compound of formula (I) or pharmaceutically acceptable salt thereof.

In another aspect, the present invention provides a pharmaceutical composition comprising a compound of formula (I) or a pharmaceutically acceptable salt thereof and a pharmaceutically acceptable carrier.

The composition of the present invention comprises at least one compound of formula (I) or a pharmaceutically acceptable salt thereof together with one or more pharmaceutically acceptable carriers and, optionally, other therapeutic agents. Compositions of the invention include those suitable for oral, rectal, nasal, topical (including buccal and sublingual), vaginal or parenteral (including subcutaneous, intramuscular, intravenous and intradermal) administration. Administration via the lungs or nasal cavity, intrathecal or intracranial injection or infusion techniques is also possible. The compositions may conveniently be presented in unit dosage form and may be prepared by methods well known in the art of pharmacy. Such methods include the step of bringing into association the active ingredient with the carrier which constitutes one or more accessory ingredients. In general, the compositions are prepared by uniformly and intimately bringing into association the compound of formula (I) or pharmaceutically acceptable salt thereof with liquid carriers, diluents, adjuvants and/or excipients or finely divided solid carriers or both, and then, if necessary, shaping the product.

The term “subject” as used herein refers to any animal. The subject may be a mammal, e.g. a human. In some embodiments, the subject is a companion animal such as a dog or cat, a domestic animal such as a horse, pony, donkey, mule, llama, alpaca, pig, cow or sheep, or a zoo animal such as a primate, felid, canid, bovid or ungulate.

As used herein, the term “therapeutically effective amount” refers to an amount of a compound effective to yield a desired therapeutic response. The specific “therapeutically effective amount” will vary with such factors as the particular condition being treated, the physical condition of the subject, the type of subject being treated, the duration of the treatment, the nature of concurrent therapy (if any), and the specific formulation employed, and the attending clinician will be able to determine an appropriate therapeutically effective amount. For example, the attending clinician may determine an appropriate therapeutically effective amount of a compound of formula (I) or a pharmaceutically acceptable salt thereof having regard to conventional dosages of other neurologically active compounds or the results of animal experiments. In some embodiments, the compound of formula (I) or pharmaceutically acceptable salt thereof may be administered at a dosage of about 1 to about 20 mg/kg body weight/day.

As used herein, a “pharmaceutically acceptable carrier” is a pharmaceutically acceptable solvent, suspending agent or vehicle for delivering a compound to a subject. The carrier may be in any form including a solid, liquid or gas and is selected with the planned manner of administration in mind. The carrier is “pharmaceutically acceptable” in the sense of being not biologically or otherwise undesirable, i.e. the carrier may be administered to a subject along with the active ingredient without causing any or a substantial adverse reaction.

The compounds of formula (I) or pharmaceutically acceptable salt thereof may be administered orally as tablets, aqueous or oily suspensions, lozenges, troches, powders, granules, emulsions, capsules, syrups or elixirs. A composition for oral use may contain one or more agents selected from the group of sweetening agents, flavouring agents, colouring agents, disintegrating agents, lubricants, time delay agents and preserving agents in order to produce pharmaceutically elegant and palatable preparations. Suitable sweeteners include sucrose, lactose, glucose, aspartame or saccharin. Suitable disintegrating agents include corn starch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable preservatives include sodium benzoate, vitamin E, alphatocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Preparations for parenteral administration are typically in the form of a sterile aqueous or non-aqueous solution, suspension or emulsion. Examples of suitable non-aqueous solvents are propylene glycol, polyethylene glycol, vegetable oils such as olive oil, and injectable organic esters such as ethyl oleate. Suitable aqueous carriers include water and alcoholic/aqueous solutions, emulsions or suspensions, including saline and buffered media. Suitable parenteral vehicles include sodium chloride solution. Preservatives and other, additives may also be present such as, for example, antimicrobials, anti-oxidants, chelating agents, growth factors, inert gases, and the like.

Generally, the terms “treating”, “treatment” and the like are used herein to mean affecting a subject to obtain a desired pharmacological and/or physiological effect. The effect may be prophylactic in terms of completely or partially preventing a disease or disorder or sign or symptom thereof, and/or may be therapeutic in terms of a partial or complete cure of a disease or disorder. “Treating” as used herein covers any treatment of, or prevention of, disease or disorder in a vertebrate, a mammal, particularly a human, and includes: (a) preventing the disease or disorder from occurring in a subject that may be predisposed to the disease or disorder, but has not yet been diagnosed as having the disease or disorder; (b) inhibiting the disease or disorder, i.e., arresting the development of the disease or disorder; or (c) relieving or ameliorating the effects of the disease or disorder, i.e. causing regression of the effects of the disease or disorder.

### EXAMPLES

Embodiments of the invention are described below by reference to the following non-limited examples.

1. General Synthesis

12 Carbon Linked Pyrazolopyrimidine Subunits

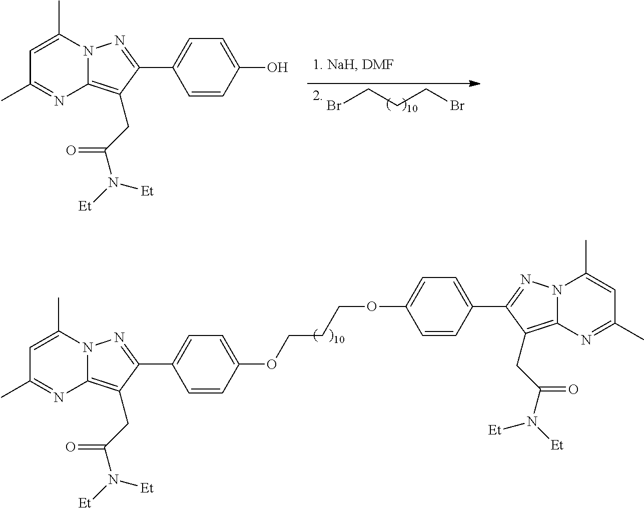

To a stirred suspension of sodium hydride (14.2 mg of a 60% w/w dispersion in oil, 0.356 mmol, 1.25 equiv.) in anhydrous dimethylformamide (1.0 mL) was added a solution of the phenol (99.5 mg, 0.284 mmol, 2.0 equiv.) in anhydrous dimethylformamide (2.0 mL) under an argon atmosphere. A bright yellow colour rapidly developed as the sodium phenoxide was formed. After 30 minutes of stirring at ambient temperature the reaction mixture was treated with a solution of 1,12-dibromododecane (46.7 mg, 0.142 mmol, 1.0 equiv.) in anhydrous dimethylformamide (1.0 mL). The reaction mixture was stirred at 100° C. for a further 36 hours after which time thin layer chromatography revealed complete conversion of the phenol starting material. The reaction mixture was partitioned between water and ethyl acetate, the organic phase was isolated and the aqueous phase was further extracted with dichloromethane. The combined organic extracts were washed with water, dried over anhydrous sodium sulfate and concentrated in vacuo. The crude product thus obtained was purified by flash column chromatography on silica gel (dichloromethane-methanol, 98:2) to give an off white solid which was triturated with hexane to afford the desired bidentate ligand as a white solid. ¹H NMR (200 MHz, CDCl₃) δ 7.75 (d, J=8.7 Hz, 4H, Ar—H), 6.97 (d, J=8.8 Hz, 4H, Ar—H), 6.49 (s, 2H, Ar—H), 3.99 (t, J=6.5 Hz, 4H), 3.91 (s, 4H), 3.55-3.35 (m, 8H, N(CH₂CH₃)₂), 2.73 (s, 6H, Ar—CH₃), 2.53 (s, 6H, Ar—CH₃), 1.83-1.73 (br m, 4H), 1.45-1.16 (br m, 16H), 1.22-1.07 (m, 12H, N(CH₂CH₃)₂); HRMS (ESI) calc'd for C₅₂H₇₀N₈O₄ (M+H⁺) 871.5593, found 871.5586, (M+Na⁺) 893.5412, found 893.5405.

8 Carbon Linked Pyrazolopyrimidine Subunits

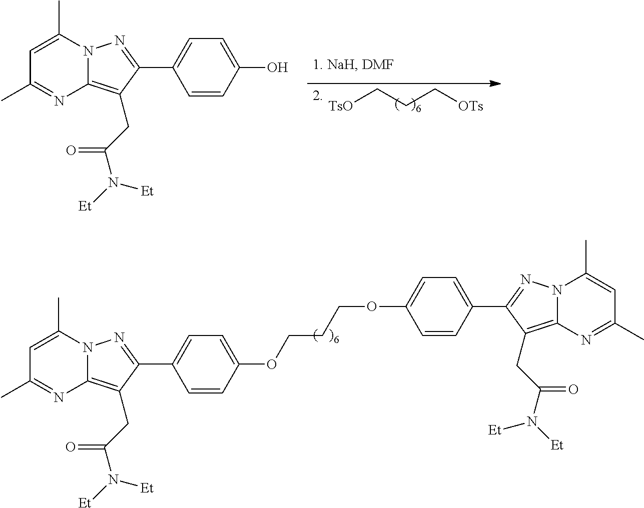

To a stirred suspension of sodium hydride (14.2 mg of a 60% w/w dispersion in oil, 0.356 mmol, 1.25 equiv.) in anhydrous dimethylformamide (1.0 mL) was added a solution of the phenol (101.2 mg, 0.284 mmol, 2.0 equiv.) in anhydrous dimethylformamide (2.0 mL) under an argon atmosphere. A bright yellow colour rapidly developed as the sodium phenoxide was formed. After 30 minutes of stirring at ambient temperature the reaction mixture was treated with a solution of the ditosylate derived from 1,8-octanediol (64.5 mg, 0.142 mmol, 1.0 equiv.) in anhydrous dimethylformamide (1.0 mL). The reaction mixture was stirred at 100° C. for a further 36 hours after which time it was partitioned between water and ethyl acetate, the organic phase was isolated and the aqueous phase was further extracted with dichloromethane. The combined organic extracts were washed with water, dried over anhydrous sodium sulfate and concentrated in vacuo to afford an off white solid. The ¹H NMR spectrum revealed a mixture of unchanged phenol and the desired bidentate. The crude mixture was redissolved in dichloromethane and washed with a 1 M aqueous solution of sodium hydroxide. The organic phase,was isolated, dried over anhydrous sodium sulfate and concentrated in vacuo to afford an off white solid which was triturated with hexane to give the desired bidentate ligand as a white solid. ¹H NMR (200 MHz, CDCl₃) δ 7.75 (d, J=8.7 Hz, 4H, Ar—H), 6.97 (d, J=8.8 Hz, 4H, Ar—H), 6.49 (s, 2H, Ar—H), 4.00 (t, J=6.3 Hz, 4H), 3.91 (s, 4H), 3.51-3.39 (m, 8H, N(CH₂CH₃)₂), 2.73 (s, 6H, Ar—CH₃), 2.53 (s, 6H, Ar—CH₃), 1.81-1.22 (br m, 6H), 1.22-1.07 (m, 12H, N(CH₂CH₃)₂); HRMS (ESI) calc'd for C₄₈H₆₂N₈O₄ (M+H⁺) 815.4967, found 815.4963, (M+Na⁺) 837.4786, found 837.4780.

6 Carbon Linked Pyrazolopyrimidine Subunits

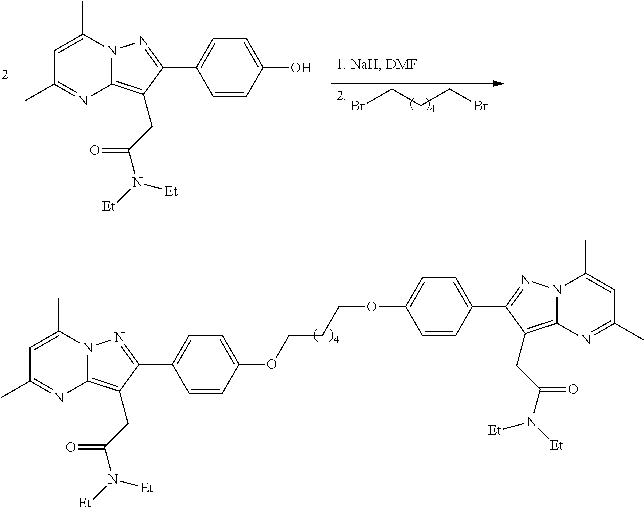

To a stirred suspension of sodium hydride (14.2 mg of a 60% w/w dispersion in oil, 0.356 mmol, 1.25 equiv.) in anhydrous dimethylformamide (1.0 mL) was added a solution of the phenol (100 mg, 0.284 mmol, 2.0 equiv.) in anhydrous dimethylformamide (2.0 mL) under an argon atmosphere. A bright yellow colour rapidly developed as the sodium phenoxide was formed. After 30 minutes of stirring at ambient temperature the reaction mixture was treated with 1,6-dibromohexane (21.6 μL, 0.142 mmol, 1.0 equiv.). The reaction mixture was stirred at 100° C. for a further 36 hours after which time it was partitioned between water and ethyl acetate, the organic phase was isolated and the aqueous phase was further extracted with dichloromethane. The combined organic extracts were washed with water, dried over anhydrous sodium sulfate and concentrated in vacuo to afford an off white solid. The ¹H NMR spectrum revealed a mixture of unchanged phenol and the desired bidentate. The crude mixture was redissolved in dichloromethane and washed with a 1 M aqueous solution of sodium hydroxide. The organic phase was isolated, dried over anhydrous sodium sulfate and concentrated in vacuo to afford an off white solid which was triturated with hexane to give the desired bidentate ligand as a white solid. ¹H NMR (200 MHz, CDCl₃) δ 7.75 (d, J=8.6 Hz, 4H, Ar—H), 6.97 (d, J=8.7 Hz, 4H, Ar—H), 6.49 (s, 2H, Ar—H), 4.06 (br m, 4H), 3.91 (s, 4H), 3.54-3.35 (m, 8H, N(CH₂CH₃)₂), 2.73 (s, 6H, Ar—CH₃), 2.53 (s, 6H, Ar—CH₃), 1.81 (br m, 4H), 1.51 (br m, 4H), 1.29-1.08 (m, 12H, N(CH₂CH₃)₂); HRMS (ESI) calc'd for C₄₆H₅₈N₈O₄ (M+H⁺) 787.4654, found 787.4669, (M+Na⁺) 809.4473, found 809.4464.

4 Carbon Linked Pyrazolopyrimidine Subunits

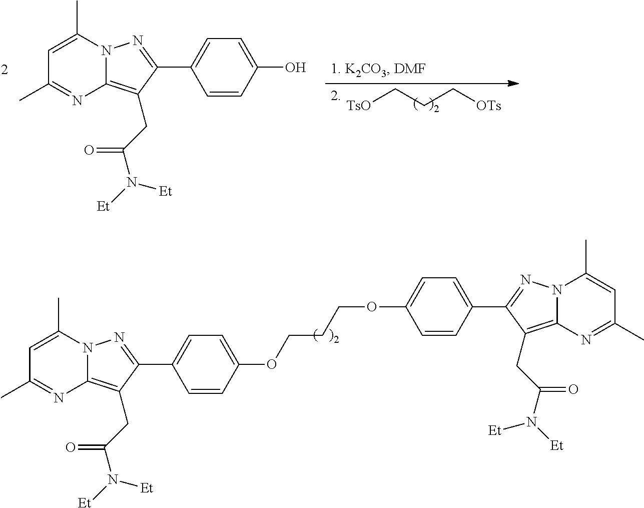

To a stirred solution of anhydrous potassium carbonate (40.9 mg, 0.284 mmol, 4.0 equiv.) and the phenol (100 mg, 0.284 mmol, 2.0 equiv.) in anhydrous dimethylformamide (2.0 mL) under an argon atmosphere was added a solution of the ditosylate derived from 1,4-butanediol (56.6 mg, 0.142 mmol, 1.0 equiv.) in anhydrous dimethylformamide (1.0 mL). The reaction mixture was stirred at 100° C. for 36 hours after which time it was partitioned between water and ethyl acetate, the organic phase was isolated and the aqueous phase was further extracted with dichloromethane. The combined organic extracts were washed with water, dried over anhydrous sodium sulfate and concentrated in vacuo to afford an off white solid. The ¹H NMR spectrum revealed a mixture of unchanged phenol and the desired bidentate. The crude mixture was redissolved in dichloromethane and washed with a 1 M aqueous solution of sodium hydroxide. The organic phase was isolated, dried over anhydrous sodium sulfate and concentrated in vacuo to afford an off white solid which was triturated with hexane to give the desired bidentate ligand as a white solid. ¹H NMR (200 MHz, CDCl₃) δ 7.76 (d, J=8.6 Hz, 4H, Ar—H), 6.99 (d, J=8.7 Hz, 4H, Ar—H), 6.50 (s, 2H, Ar—H), 4.06 (br m, 4H), 3.91 (s, 4H), 3.55-3.35 (m, 8H, N(CH₂CH₃)₂), 2.74 (s, 6H, Ar—CH₃), 2.53 (s, 6H, Ar—CH₃), 2.03 (br s, 4H), 1.29-1.08 (m, 12H, N(CH₂CH₃)₂); HRMS (ESI) calc'd for C₄₄H₅₄N₈O₄ (M+H⁺) 759.4341, found 759.4347, (M+Na⁺) 781.4160, found 781.4152.

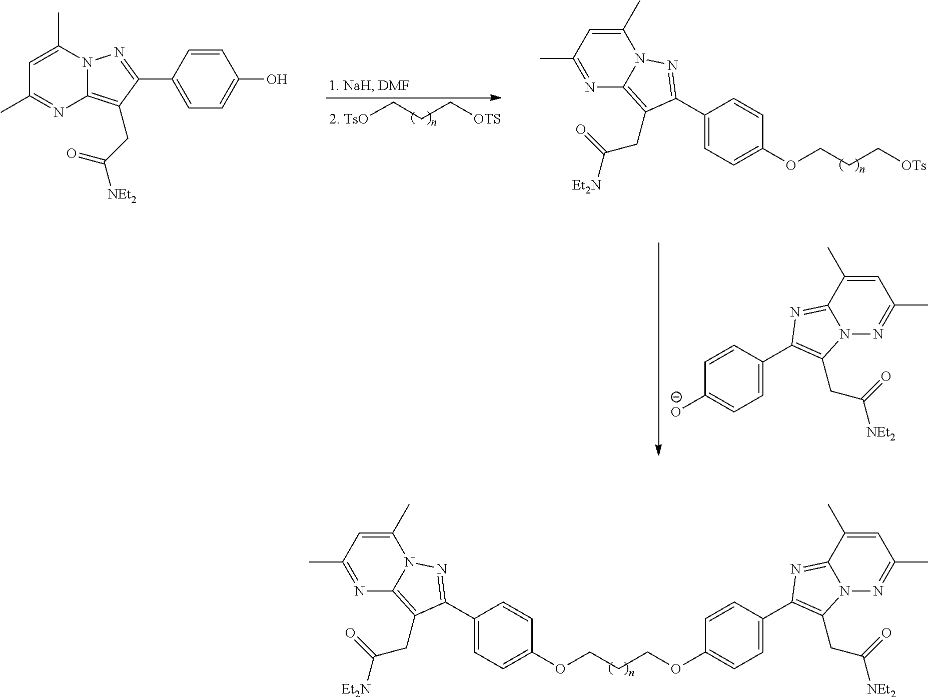

General procedure for the synthesis of heteromeric bidentates, i.e, those compounds where ligands X and Y are different. This example scheme shows a pyrazolopyrimidine ligand linked to a pyridazine ligand. It is possible for ‘n’ to be any suitable linker, for example 0 to 18.

To a stirred solution of the phenol (ligand X, 1 equiv.) in anhydrous DMF is added sodium hydride to generate the phenoxide. To this solution is added a solution of the dibromide or ditosylate substituted linker of chosen length (1 equiv.). The reaction is monitored by thin layer chromatography until such time that no starting phenol remains. The monosubstituted product is isolated and purified in the standard fashion and this material forms the starting material for the second step. To a stirred solution of the phenol (ligand Y, 1 equiv.) in anhydrous DMF is added sodium hydride to generate the phenoxide. To this solution is added a solution of the monosubstituted compound from step 1 (1 equiv.) in anhydrous DMF. The reaction is monitored by thin layer chromatography until such time that no phenol (ligand Y) remains and the product is isolated and purified in the usual manner to give the heteromeric bidentate compound.

2. Radiolabelling with [¹⁸F]

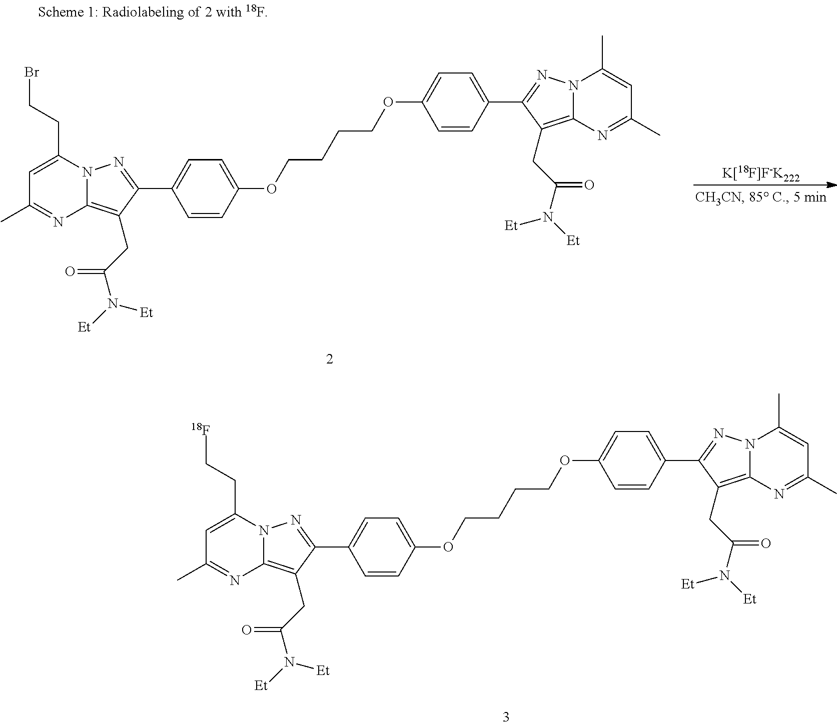

Radioisotope production. Aqueous [¹⁸F]fluoride ion can be produced on a PET trace cyclotron (GE Healthcare, Sweden), by irradiation of a 0.8 mL water target using a 16.5 MeV proton beam on 95% enriched [¹⁸O]—H₂O by the [¹⁸O(p,n)¹⁸F] nuclear reaction.

Preparation of [¹⁸F]-kryptofix-K222. In a typical radiofluorination reaction, [¹⁸F]Fluoride in [¹⁸O] enriched-H₂O is transferred to a GE TRACERlab MXFDₒ synthesiser and passed through an anion exchange resin (Sep-Pak Waters Accell™ Light QMA cartridge in the carbonate form, made by washing with 10 mL 0.5 M K₂CO₃ and then rinsing with 10 mL of water) under vacuum. Trapped [¹⁸F]fluoride ions are then eluted from the Sep-Pak cartridge and transferred to the reactor vessel using an eluent solution containing K₂CO₃ (7 mg in 300 μL of pure water), 300 μL of acetonitrile and 22 mg of Kryptofix 222 (K222: 4,7, 13,16,2 l,24-hexaoxa-l,10-diazabicyclo [8.8.8]hexacosan). Aliquots of acetonitrile are added and the reaction mixture evaporated to dryness after each addition. (3 times: 80 μL, each time). The evaporation is carried out at 95° C. under nitrogen flow and vacuum.

Preparation and formulation of [¹⁸F]-3. Compound 2 is dissolved in 3 mL of acetonitrile and is added to the dry ₁₈F]-kryptofix-K222 complex. The mixture is allowed to react at 85° C. for 5 minutes. Upon completion the reaction mixture is diluted with Waters for Injections BP (WFI BP) and is passed through a tC-18 Sep-Pak cartridge. The reactor vessel is rinsed, with WFI and again is passed through the tC18 Sep-Pak cartridge. The tC18 trapped radiolabeled product is rinsed a further three times with WFI (40 mL total). The product is then eluted from the tC18 Sep-Pak cartridge. The resulting solution is passed though a 0.22 μm Millipore CATHIVEX non-pyrogenic sterile filter to remove particulate material before HPLC purification. The crude mixture is then injected onto a HPLC Waters XTerra RP C-18 IO μm (7.8×300 mm) semi-preparative reversed-phase column and eluted. The radioactive fraction corresponding to [¹⁸F]-3 is collected and is evaporated under vacuum. The residue is reconstituted in WFI BP (4 mL) and filtered through a sterile 13 mm Millipore GV 0.22 μm filter into a sterile pyrogen free evacuated vial.

Radioligand Binding Experiments Using [³H]PK11195

Cell Culture and Membrane Preparation

Human embryonic kidney cells (HEK293) were transfected with human TSPO as described previously (Riond, J., Mattei, M. G., Kaghad, M., Dumont, X., Guillemot, J. C., Le Fur, G., Caput, D., Ferrara, P. (1991) Molecular cloning and chromosomal localization of a human peripheral-type benzodiazepine receptor. Eur. J. Biochem. 195, 305-311; Vin, V., Leducq, N., Bono, F., Herbert, J. M. (2003) Binding characteristics of SSR180575, a potent and selective peripheral benzodiazepine receptor ligand. Biochem. Biophys. Res. Comm. 310, 785-790). Cells were cultured in Dulbecco's modified Eagle's medium (DMEM), supplemented with 10% foetal bovine serum 4500 mg/L D-glucose, 4 mM L-glutamine, and 100 U/ml penicillin/streptomycin. Cell cultures were maintained at 37° C. in a humidified incubator under 5% CO₂. In order to harvest cells for radioligand binding experiments, cells were first washed with pre-warmed PBS, and harvested with 0.5% PBS-EDTA, before being centrifuged at 1000 rpm for 4 minutes.

The mitochondrial fraction of the cells was obtained by homogenising the cell pellet in three volumes of 50 mM Tris-HCl (pH 7.5), containing 0.33 M sucrose, 1 mM MgCl₂, and 25 mM KCl (Solution, 1). The homogenate was centrifuged for 10 minutes at 700×g, at 4° C. The pellet was then discarded and supernatant centrifuged at 10,000×g for 10 minutes at 4° C. to yield raw mitochondria. This was purified by discarding the supernatant and resuspending the pellet in 3 volumes of Solution 1, and centrifuging at 20,000×g for 10 minutes at 4° C. to yield a pellet consisting of pure mitochondria. The resultant pellet was, then resuspended in an appropriate amount of reaction buffer (50 mM Tris-HCl, pH 7.5), and protein concentration determined using a Bio-Rad Lowry Protein Assay Kit. Samples were stored in aliquots at −20° C. until use in binding assays.

[³H]PK11195 Competition, Binding Assay

On the day of experimentation, membranes were resuspended in 50 mM Tris-HCL buffer (pH 7.5). Membranes containing a final concentration of approximately 40 μg/ml of protein were incubated with 6 nM [³H]PK11195 in a final reaction volume of 200 μl for 90 minutes at 4° C. Incubation occurred in the presence of a range of ligand concentrations (0.1-1000 nM) to yield dose-response curves depicting the dose-dependent displacement of [³H]PK11195 by the test compound. Compounds were compared with control samples, which consisted of vehicle alone; 2% DMSO in 50 mM Tris-HCl buffer (pH 7.5). Non-specific binding was defined in the presence of 1 μM cold PK11195, and amounted to 5-15% of total binding.

After incubation, assays were terminated by rapid filtration through a 96-well filter plate in ice-cold incubation buffer (50 mM Tris-HCl, pH 7.5), and washed 10 times with 200 μl of ice-cold incubation buffer, using a Brandel 96-sample vacuum harvester. The base of the filter plate was then sealed off and approximately 20 μl scintillation cocktail was added to each well. The top of the plate was sealed and filters were soaked in scintillation cocktail overnight at room temperature. Bound radioactivity was obtained as counts per minute (CPM), as measured using a TriLux MicroBeta scintillation counter (PerkinElmer), with a counting time of 1 minute per well. At least three independent experiments for each compound were carried out in duplicate. Results were ultimately expressed as a percentage of the specifically bound control, whereby specific binding=total binding−non-specific binding. Data was analysed and fit to a curve using GraphPad Prism 5.0.

Radioligand Binding Results

TABLE 1
Binding affinities of bidentate ligands and cold PK11195 in
competition with 6 nM [³H]PK11195 in HEK293 cells transfected
with human TSPO. Binding data is fit to one of two curves; one-site
competition versus two-site competition, indicated by the Kᵢ value(s).
The structures of L-4-L, L-6-L, L-8-L and L-12-L are as shown below.

Binding Affinity (Kᵢ) ± Standard Error (nM)

| Compound | Site 1 | Site 2 |
|---|---|---|
| PK11195 | 6.082 ± 0.330 | N/A |
| L-4-L | 5.991 ± 0.470 | N/A |
| L-6-L | 0.009 ± 0.012 | 11.54 ± 1.28 |
| L-8-L | 0.332 ± 0.260 | 51.34 ± 13.06 |
| L-12-L | 0.052 ± 0.076 | 48.73 ± 11.81 |

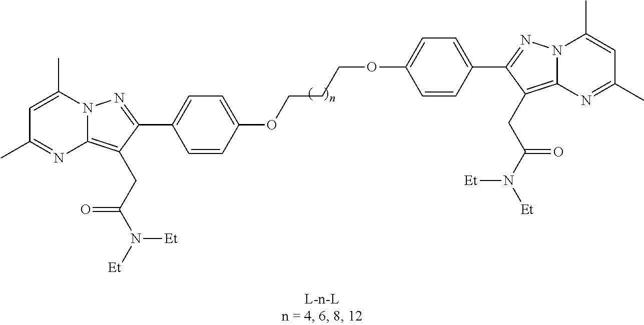

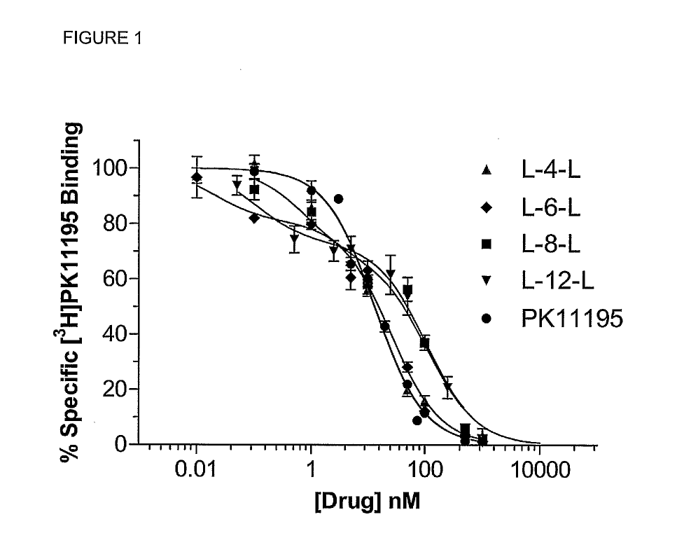

The dose response curves are shown in FIG. 1, which depict the dose-dependent displacement of [³H]PK11195 binding in HEK293 cells transfected with human TSPO, in the presence of various bidentate ligands at concentrations ranging from 0.01 nM to 1 μM. Binding data is fit to one of two curves; one-site competition versus two-site competition.

Although the invention has been described with reference to specific examples, it will be appreciated by those skilled in the art that the invention may be embodied in many other forms.

1. A compound of formula (I)
X-L-Y (I)
wherein,
X and Y independently bind TSPO, wherein X and Y are the same or different; and
L is a linker that links X to Y;
or a salt or solvate thereof.

2. The compound according to claim 1 wherein X and Y are independently selected from 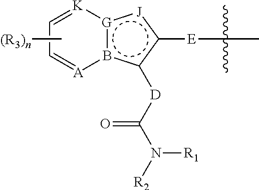
wherein,
A and K are independently CH, C or N, J is CH or N, and B and G are independently C or N provided that at least one of B and G is C, wherein at least two of A, B, G, J and K are N;
D is O, NH, (CH₂)ₘ or S;
E is an aryl group or a heteroaryl group optionally substituted with one or more of the following substituents: halogen, C₁-C₁₀ alkyl, C₂-C₁₀ alkenyl, C₂-C₁₀ alkynyl, TC₁-C₆ alkyl, TC₂-C₁₀ alkenyl, or TC₂-C₁₀ alkynyl, each of which is optionally substituted with one or more halogen substituents, and wherein T is NH, O or S;
R₁ and R₂ are independently hydrogen, C₁-C₁₀ alkyl, C₂-C₁₀ alkenyl, C₂-C₁₀ alkynyl, aryl or heteroaryl, each being optionally substituted with one of more halogen;
or R₁ and R₂ together with the nitrogen to which they are attached, form a heterocylic ring having between 3 and 7 ring members, optionally substituted with one of more halogen;
R₃ is independently halogen, C₁-C₁₀ alkyl, C₂-C₁₀ alkenyl, C₂-C₁₀ alkynyl, TC₁-C₆ alkyl, TC₂-C₁₀ alkenyl or TC₂-C₁₀ alkynyl, each of which is optionally substituted with one or more halogen substituents, and wherein T is NH, O or S;
m is a number between 1 and 6; and
n is a number between 0 and 3.

3. The compound according to claim 2 wherein
A, G and J are N, K is CH or C and B is C; or
A, B and J are N, K is CH or C and G is C.

4. The compound according to claim 2 wherein R₃ is a Cᵢ-C₆ alkyl, and wherein n is 1 or 2.

5. The compound according to claim 2 wherein n is 2 and each respective R₃ is methyl.

6. The compound according to claim 5 wherein the respective methyl groups are positioned meta to each other.

7. The compound according to claim 2 wherein D is (CH₂)ₘ, and wherein m is 1.

8. The compound according to claim 2 wherein R₁ and R₂ are independently a C₁-C₆ alkyl.

9. The compound according to claim 2 wherein R₁ and R₂ are independently ethyl.

10. The compound according to claim 2 wherein E is a 5-, or 6-membered aryl or heteroaryl group optionally substituted with one or more substituents selected from the group consisting of halogen, C₁-C₆ alkyl, C₂-C₆ alkenyl, and C₂-C₆ alkynyl.

11. The compound according to claim 2 wherein E is phenyl.

12. The compound according to claim 2 wherein X and Y are independently

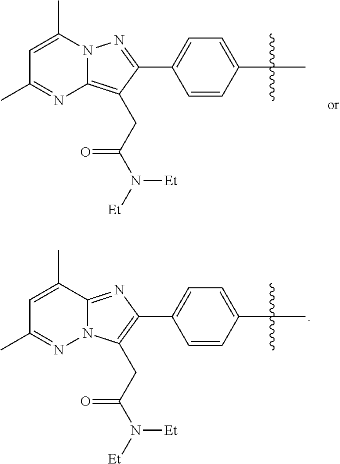

13. The compound according to claim 1 wherein L is selected from the group consisting of C₁-C₂₀ alkyl, C₂-C₂₀ alkenyl, C₂-C₂₀ alkynyl, T(C₁-C₂₀ alkyl)T, T(C₂-C₂₀ alkenyl)T, T(C₂-C₂₀ alkynyl)T, TCH₂(CH₂OCH₂)ₚCH₂T;
TCH₂(CH₂NHCH₂)ₚCH₂T, amino acids comprising glycine oligimers; wherein T is NH, O or S; and
wherein p is a number between 1 and 10.

14. The compound according to claim 13 wherein L is selected from the group consisting of O(C₁-C₂₀ alkyl)O, O(C₂-C₂₀ alkenyl)O, O(C₂-C₂₀ alkynyl)O and OCH₂(CH₂OCH₂)ₚCH₂O; and
wherein p is a number between 1 and 10.

15. The compound according to claim 2 selected from the group consisting of:

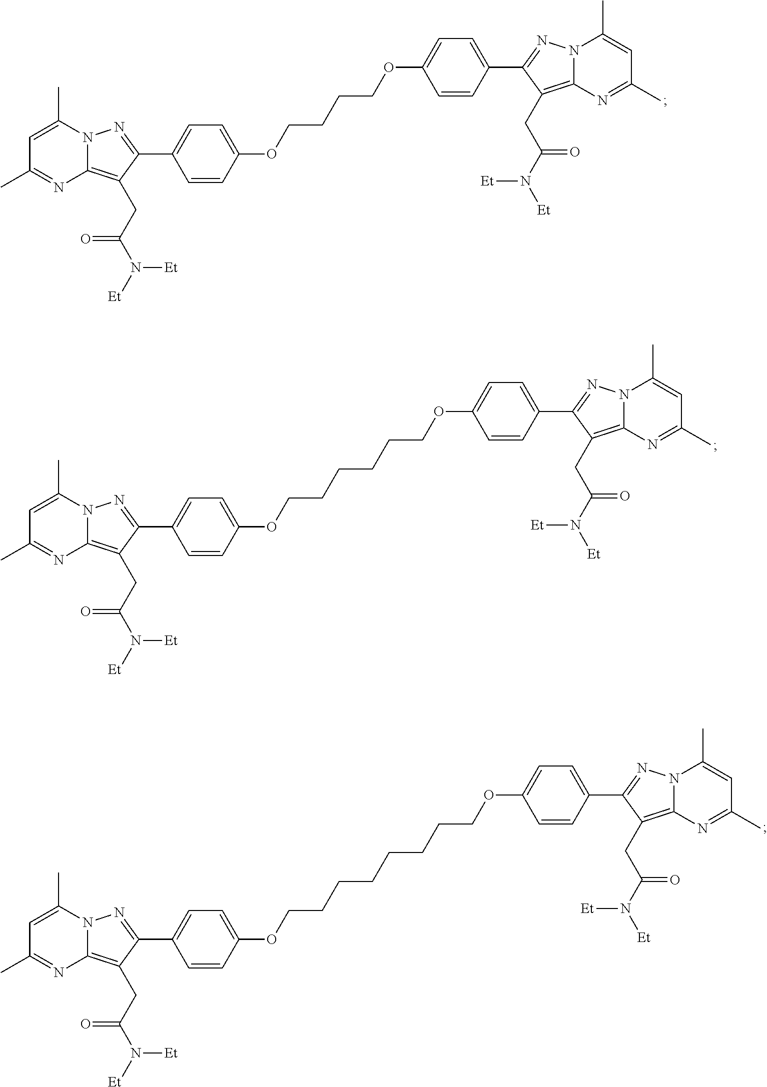

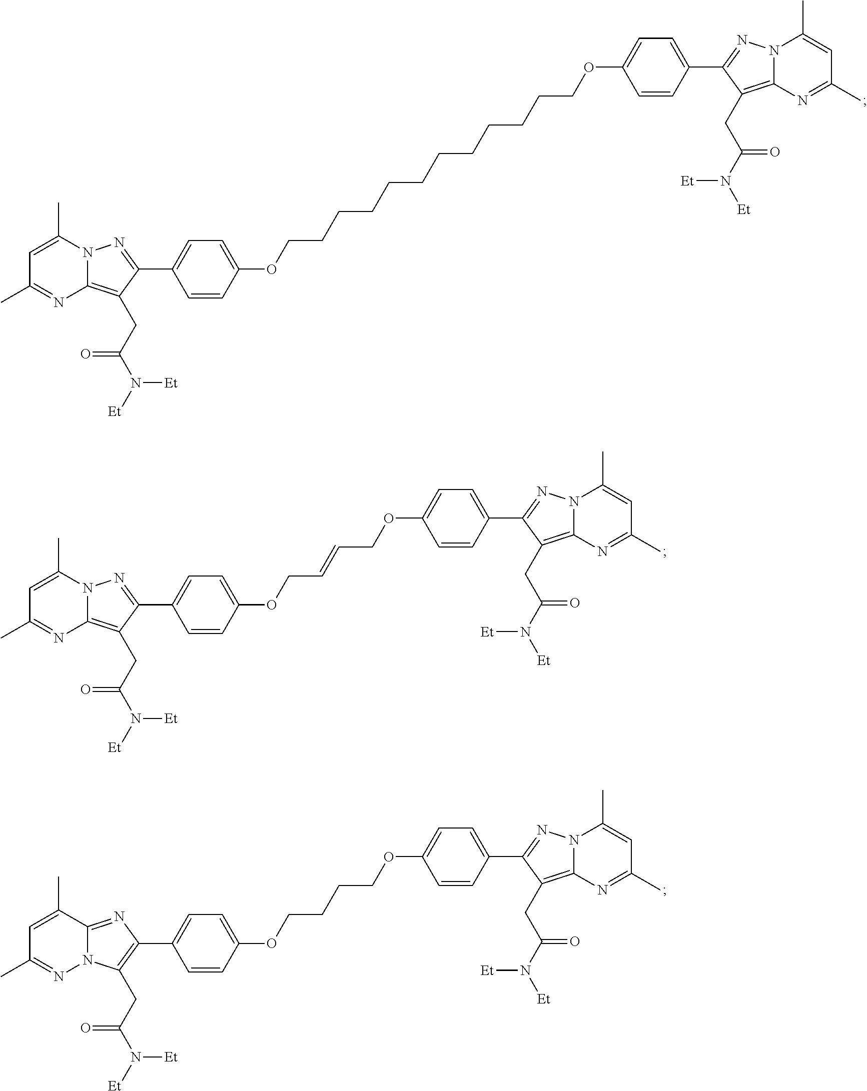

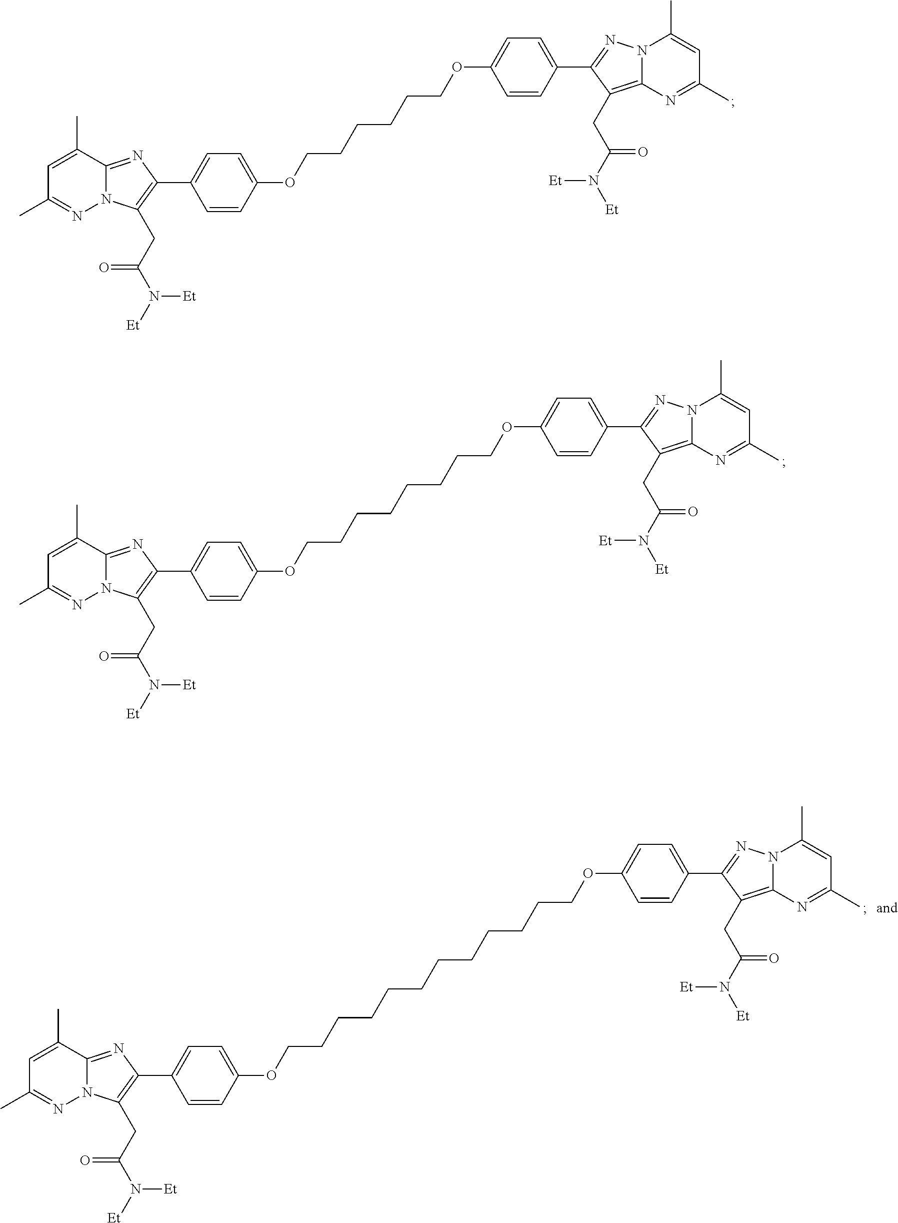

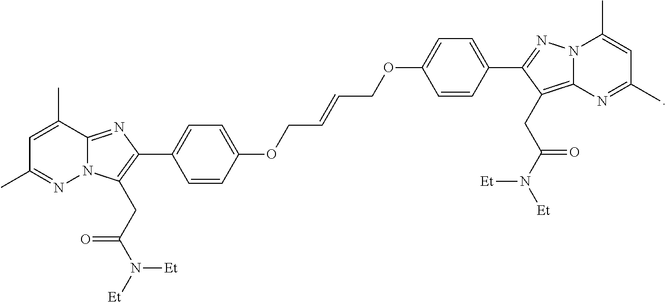

16. (canceled)

17. The compound of formula (I) according to claim 1 radiolabelled with a radioisotope.

18. The compound according to claim 17 wherein said radioisotope is selected from the group consisting of ¹⁸F, ¹²³I, ⁷⁶Br, ¹²⁴I and ⁷⁵Br.

19. The compound according to claim 18 wherein said radioisotope is ¹⁸F.

20. A pharmaceutical composition comprising a compound according to claim 1, or a pharmaceutically acceptable salt thereof, and a pharmaceutically acceptable carrier.

21. A method of diagnosing a disorder in a subject, comprising administering to the subject a compound of formula (I) according to claim 1.

22. The method according to claim 21 wherein the method comprises imaging translocator protein (18 kDa) (TSPO) in the subject.

23. The method according to claim 21 wherein, when the compound is radiolabelled with a radioisotope, said radioisotope is selected from the group consisting of ¹⁸F, ¹²³1, ¹²⁴I, ⁷⁵Br and ⁷⁶Br.

24. The method according to claim 22, wherein the method comprises obtaining an image indicating the location of the protein.

25. The method according to claim 24 wherein the image is obtained by positron emission tomography (PET) imaging.

26. The method according to claim 24 wherein the compound of formula (I) is radiolabelled with ¹²³I and the image is obtained by SPECT imaging.

27. The method according to claim 24 any one of claims 2 wherein said image is obtained to assess the extent of TSPO binding of the compound or salt thereof in the brain parenchyma of the subject.

28. The method according to claim 21 wherein the disorder is a neurodegenerative disorder, inflammation or anxiety.

29. The method according to claim 21 wherein the disorder is selected from the group consisting of: Alzheimer's disease, Parkinson's disease, Huntington's disease, multiple sclerosis, multiple system atrophy, epilepsy, encephalopathy, stroke, brain tumour, anxiety, stress, emotional disturbances or cognitive impairment, glioblastoma, ischemic stroke, herpes encephalitis, HIV, amyotrophic lateral sclerosis, corticobasal degeneration, cancer, depression, an auto-immune disease and an infectious disease.

30. The method according to claim 21 wherein the subject is a human.

31-39. (canceled)

40. A method of treating a disorder in a subject comprising administering to the subject a compound according to claim 1.

41. The method according to claim 40 wherein the disorder is characterized by an abnormal density of TSPO receptors in a mammal.

42. The method according to claim 40 wherein the disorder is a neurodegenerative disorder, inflammation or anxiety in a subject.

43. The method of claim 40 wherein the disorder is Alzheimer's disease, Parkinson's disease, Huntington's disease, multiple sclerosis, multiple system atrophy, epilepsy, encephalopathy, stroke, brain tumour, anxiety, stress, emotional disturbances or cognitive impairment, glioblastoma, ischemic stroke, herpes encephalitis, HIV, amyotrophic lateral sclerosis, corticobasal degeneration, cancer, depression, auto-immune and infectious diseases.

44. A process for preparing a compound of formula (I), said process comprising reacting a compound of formula (II) with V-L-V in the presence of a base 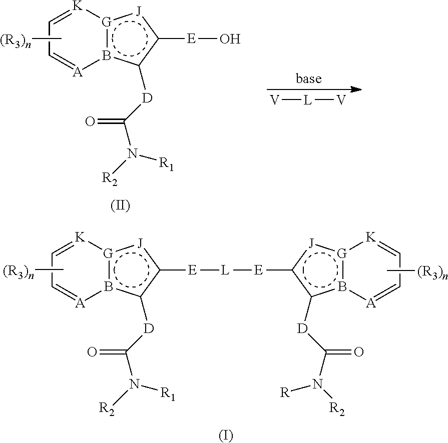
wherein,
A and K are independently CH, C or N, J is CH or N, and B and G are independently C or N provided that at least one of B and G is C, wherein at least two of A, B, G, J and K are N;
D is O, NH, (CH₂)ₘ or S;
E is an aryl group or a heteroaryl group optionally substituted with one or more of the following substituents: halogen, C₁-C₁₀ alkyl, C₂-C₁₀ alkenyl, C₂-C₁₀ alkynyl, TC₁-C₆ alkyl, TC₂-C₁₀ alkenyl, or TC₂-C₁₀ alkynyl, each of which is optionally substituted with one or more halogen substituents, and wherein T is NH, O or S;
R₁ and R₂ are independently hydrogen, C₁-C₁₀ alkyl, C₂-C₁₀ alkenyl, C₂-C₁₀ alkynyl, aryl or heteroaryl, each being optionally substituted with one of more halogen;
or R₁ and R₂ together with the nitrogen to which they are attached, form a heterocylic ring having between 3 and 7 ring members, optionally substituted with one of more halogen;
R₃ is independently halogen, C₁-C₁₀ alkyl, C₂-C₁₀ alkenyl, C₂-C₁₀ alkynyl, TC₁-C₆ alkyl, TC₂-C₁₀ alkenyl or TC₂-C₁₀ alkynyl, each of which is optionally substituted with one or more halogen substituents, and wherein T is NH, O or S;
m is a number between 1 and 6; and
n is a number between 0 and 3;
L is selected from the group consisting of C₁-C₂₀ alkyl, C₂-C₂₀ alkenyl, C₂-C₂₀ alkynyl, T(C₁-C₂₀ alkyl)T, T(C₂-C₂₀ alkenyl)T, T(C₂-C₂₀ alkynyl)T, TCH₂(CH₂OCH₂)ₚCH₂T;
TCH₂(CH₂NHCH₂)ₚCH₂T, amino acids including but not limited to glycine oligimers; wherein T is NH, O or S;
wherein p is a number between 1 and 10;
wherein V is a leaving group that reacts with a base; and
wherein the base is NaH or K₂CO₃.

45. A compound of formula (I) according to claim 1 capable of eliciting a response when bound to a TSPO receptor.
